(19) United States
(12) Patent Application Publication
(10) Pub. No.: US 2024/0044833 A1
(43) Pub. Date: Feb. 8, 2024

(54) ELECTROCHEMICAL BIOSENSOR FOR TARGET ANALYTE DETECTION

(71) Applicant: McMaster University, Hamilton (CA)

(72) Inventors: Leyla Soleymani, Oakville (CA); Sarah Traynor, Hamilton (CA); Feng Li, St. Catharines (CA)

(21) Appl. No.: 18/026,043

(22) PCT Filed: Sep. 14, 2021
(86) PCT No.: PCT/CA2021/051281

§ 371 Date: Mar. 13, 2023

Related U.S. Application Data
(60) Provisional application No. 63/077,965, filed on Sep. 14, 2020.

(51) Int. Cl. G01N 27/327; G01N 33/53; G01N 33/543

(52) U.S. Cl. CPC G01N 27/3276; G01N 33/5308; G01N 33/5438; G01N 27/3277

(57) ABSTRACT

This disclosure relates to a biosensor for detecting a target analyte in a sample comprising: a double-stranded oligonucleotide comprising an overhang on a first strand of the oligonucleotide, and a second strand of the oligonucleotide that is a reporter moiety comprising a detectable label; a first detection probe comprising a recognition moiety and a junction forming moiety, wherein the junction forming moiety comprises a first portion capable of binding by complementarity to the overhang of the first strand of the double-stranded oligonucleotide; a second detection probe comprising a recognition moiety and a junction forming moiety, wherein the junction forming moiety comprises a first portion capable of binding by complementarity to an internal segment of the first strand of the double-stranded oligonucleotide; and a capture probe functionalized on an electrode, wherein the capture probe comprises an immobilized strand attached to the electrode. Methods and uses thereof are also disclosed herein.

### RELATED APPLICATION

This disclosure claims benefit of U.S. Provisional Patent Application Ser. No. 63/077,965 filed Sep. 14, 2020, incorporated herein by reference in its entirety.

### INCORPORATION OF SEQUENCE LISTING

The sequence listing that is contained in the file named “P62723PC00_ST25”, which is 1,968 bytes and was created on Sep. 14, 2021, is filed herewith by electronic submission and is incorporated by reference herein.

### FIELD

The present disclosure relates to biosensors, and in particular, to electrochemical biosensors and methods for target analyte detection.

### BACKGROUND

Sensitive and accurate protein analysis is critical to disease diagnostics, monitoring, and management. The existing laboratory-scale instruments for protein analysis do not allow for frequent screening and monitoring of patients at primary healthcare settings, patient bedsides, or home settings mainly due to their high cost and complex operating protocols for non-technical users. To develop a point-of-care (POC) protein analyzer, major research efforts are targeted toward developing sensitive and specific biosensors that parallel the handheld glucose monitor in terms of ease-of-operation, response time, and operating and equipment cost.

Electrochemical readout is ideally-suited for POC protein biosensing because it offers high detection sensitivity with rapid readout and is compatible with low-cost and miniaturized readout circuitry. However, most electrochemical protein biosensors fail to operate in a sample-in-answer-out (SIAO) manner, especially when they are challenged with unprocessed clinical samples. This difficulty stems from the dependence of these assays on multiple steps involving washes, target labeling, and the addition of reagents to process the sample and amplify and transduce a signal.

Programmable DNA-based assays—target-responsive structure switching assays (DNAzymes and aptamer), proximity-dependent surface hybridization assays proximity ligation assays (PLA), and nucleic acid programmable protein arrays (NAPPA)—have been used to integrate protein capture with built-in signal transduction to eliminate the need for multi-step processing. Among these methods, bio-barcode assays that generate a nucleic acid barcode in response to protein recognition hold great promise for simplifying protein analysis. In spite of this, the common employment of loaded nanoparticles (NPs) or enzymes for amplifying the nucleic acid reporter, makes single-step operation using these assays challenging.

The background herein is included solely to explain the context of the disclosure. This is not to be taken as an admission that any of the material referred to was published, known, or part of the common general knowledge as of the priority date.

### SUMMARY

The present disclosure describes an electrochemical bio-barcode assay (e-biobarcode assay) that integrates biorecognition with signal transduction using molecular (for example, DNA/protein) machines and signal readout using, for example nanostructured, electrodes. The e-biobarcode assay eliminates multi-step processing and uses a single step for analysis following sample collection into, for example, a reagent tube.

According to an aspect of the present disclosure, there is provided a biosensor for detecting a target analyte in a sample comprising:
a) a double-stranded oligonucleotide comprising an overhang on a first strand of the oligonucleotide, and a second strand of the oligonucleotide that is a reporter moiety comprising a detectable label;
b) a first detection probe comprising a recognition moiety and a junction forming moiety, wherein the junction forming moiety comprises a first portion capable of binding by complementarity to the overhang of the first strand of the double-stranded oligonucleotide;
c) a second detection probe comprising a recognition moiety and a junction forming moiety, wherein the junction forming moiety comprises a first portion capable of binding by complementarity to an internal segment of the first strand of the double-stranded oligonucleotide; and
d) a capture probe functionalized on an electrode, wherein the capture probe comprises an immobilized strand attached to the electrode, and optionally a displaceable strand binding to the immobilized strand by partial complementarity;
wherein the junction forming moiety of the first detection probe comprises a second portion complementary to a second portion of the junction forming moiety of the second detection probe,
wherein in the presence of the target analyte, the recognition moiety of the first detection probe binds to the target analyte, and the recognition moiety of the second detection probe binds to a different portion of the target analyte, thereby bringing the first detection probe and the second detection probe into proximity sufficient to allow for the second portion of the first detection probe to bind by complementarity to the second portion of the second detection probe, whereby the first detection probe and the second detection probe form a stable duplex,
wherein in the presence of the target analyte, the first portion of the first detection probe binds by complementarity to the overhang of the first strand of the double-stranded oligonucleotide, and the first portion of the second detection probe binds by complementarity to the internal segment of the first strand of the double-stranded oligonucleotide, whereby the binding of the first detection probe and the second detection probe to the first strand of the double-stranded oligonucleotide releases the reporter moiety from the double-stranded oligonucleotide in a), and
wherein the released reporter moiety is capable of binding by complementarity to the immobilized strand of the capture probe attached to the electrode, whereby the binding is capable of displacing the optional displaceable strand and bringing the detectable label on the reporter moiety close to the electrode surface for producing a detectable electrochemical signal.

In some embodiments, the recognition moiety of the first detection probe and the recognition moiety of the second detection probe, each independently, comprises a nucleic acid, a small molecule, a peptide, or a protein. In some embodiments, the protein is an antibody or an antigen-binding fragment thereof. In some embodiments, the junction forming moiety of the first detection probe and the junction forming moiety of the second detection probe, each independently, comprises a nucleic acid. In some embodiments, the immobilized strand of the capture probe and the displaceable strand of the capture probe, each independently, comprises a nucleic acid.

In some embodiments, the detectable label comprises a redox species or photoelectrochemical species. In some embodiments, the detectable label comprises a redox species. In some embodiments, the redox species is selected from the group consisting of methylene blue, methylene blue succinimide, methylene blue maleimide, Atto MB2 maleimide, other methylene blue derivatives, 3,7-Bis-[(2-Ammoniumethyl) (methyl)amino]phenothiazin-5-ium trifluoroacetate, 3,7-Bis-(piperazin-4-ium-1-yl)phenothiazin-5-ium trifluoroacetate, 3,7-Bis-[(2-ammoniumethyl)(methyl)amino]phenothiazin-5-ium chloride, 3,7-Bis-(piperazin-4-ium-1-yl)phenothiazin-5-ium chloride, and ferrocene. In some embodiments, the redox species is methylene blue.

In some embodiments, the detectable electrochemical signal is a change in current, voltage or impedance. In some embodiments, the detectable electrochemical signal is an increase in current compared to in the absence of the target analyte. In some embodiments, the electrode comprises a conductive material, a semi-conductive material, or combinations thereof. In some embodiments, the electrode comprises a metal, a metal alloy, a metal oxide, a superconductor, a semi-conductor, a carbon-based material, a conductive polymer, or combinations thereof. In some embodiments, the electrode comprises a metal. In some embodiments, the electrode comprises three-dimensional nanostructures.

In some embodiments, the biosensor further comprises a surface blocker functionalized on the electrode. In some embodiments, the surface blocker comprises a poly-A oligonucleotide and/or mercaptohexanol. In some embodiments, the surface blocker comprises poly-A oligonucleotide and mercaptohexanol.

In some embodiments, the biosensor further comprises a counter electrode and/or a reference electrode. In some embodiments, the sample is an aqueous solution. In some embodiments, the target analyte is a protein. In some embodiments, the target analyte is prostate specific antigen. In some embodiments, the biosensor is for use in clinical and agricultural diagnostics, agri-food quality control, environmental monitoring, health screening, health monitoring, and/or pharmaceutical development.

Also provided is a method of detecting a target analyte in a sample, the method comprising:
a) mixing, optionally in a solution, the sample with the double-stranded oligonucleotide, a first detection probe and a second detection probe from the biosensor described herein, to provide a mixture, wherein upon the mixing in the presence of the target analyte, a recognition moiety of the first detection probe binds to the target analyte, a recognition moiety of the second detection probe binds to a different portion of the target analyte, thereby bringing the first detection probe and the second detection probe into close proximity sufficient to allow for the second portion of the first detection probe to bind by complementarity to the second portion of the second detection probe, thereby forming a stable duplex, and wherein upon the forming of the stable duplex, the first portion of the first detection probe binds by complementarity to an overhang of the first strand of the double-stranded oligonucleotide, and the first portion of the second detection probe binds by complementarity to an internal segment of the first strand of the double-stranded oligonucleotide, thereby forming a junction and releasing a reporter moiety from the double-stranded oligonucleotide;
b) contacting the mixture with a capture probe functionalized on an electrode, wherein upon the contacting, in the presence of the target analyte, the released reporter moiety binds to an immobilized strand of the capture probe, optionally if a displaceable strand is present, displacing the displaceable strand, and bringing a detectable label on the reporter moiety close to the electrode surface, thereby producing a detectable electrochemical signal; and
c) measuring the detectable electrochemical signal produced from the electrode.

In some embodiments, the detectable electrochemical signal is a change in current, voltage or impedance in the presence of the target analyte compared to in the absence of the target analyte. In some embodiments, the detectable electrochemical signal is an increase in current compared to in the absence of the target analyte. In some embodiments, the target analyte is a protein. In some embodiments, the protein is prostate specific antigen.

Also provided is a system comprising the biosensor described herein, and an ammeter, a voltameter, or an impedance analyzer.

Also provided is a kit for detecting a target analyte in a sample, wherein the kit comprises:
a) a double-stranded oligonucleotide comprising an overhang on a first strand of the oligonucleotide, and a second strand of the oligonucleotide that is a reporter moiety comprising a detectable label;
b) a first detection probe comprising a recognition moiety and a junction forming moiety, wherein the junction forming moiety comprises a first portion capable of binding by complementarity to the overhang of the first strand of the double-stranded oligonucleotide;
c) a second detection probe comprising a recognition moiety and a junction forming moiety, wherein the junction forming moiety comprises a first portion capable of binding by complementarity to an internal segment of the first strand of the double-stranded oligonucleotide; and
d) instructions for use.

In some embodiments, the kit further comprises a capture probe functionalized on an electrode, wherein the capture probe comprises an immobilized strand attached to the electrode, and optionally a displaceable strand binding to the immobilized strand by partial complementarity, and optionally further comprising at least one of a solution, a sample collector, a liquid dropper, a lancet, a bandage, gloves, and a mask.

Also provided is a kit for detecting a target analyte in a sample, wherein the kit comprises components required for a method described herein and instructions for use of the kit.

Also provided is a biosensor for detecting a target analyte in a sample comprising:
a) a double-stranded oligonucleotide comprising an overhang on a first strand of the oligonucleotide, and a second strand of the oligonucleotide that is a reporter moiety;
b) a first detection probe comprising a recognition moiety and a junction forming moiety, wherein the junction forming moiety comprises a first portion capable of binding by complementarity to the overhang of the first strand of the double-stranded oligonucleotide;
c) a second detection probe comprising a recognition moiety and a junction forming moiety, wherein the junction forming moiety comprises a first portion capable of binding by complementarity to an internal segment of the first strand of the double-stranded oligonucleotide; and
d) a capture probe, optionally functionalized on a solid support, wherein the capture probe comprises a signaling strand and a displaceable strand binding to the signaling strand by partial complementarity, wherein the signaling strand comprises a quenchable detectable label and the displaceable strand comprises a quencher in sufficiently close proximity to and capable of quenching the quenchable detectable label; wherein the junction forming moiety of the first detection probe comprises a second portion complementary to a second portion of the junction forming moiety of the second detection probe,
wherein in the presence of the target analyte, the recognition moiety of the first detection probe binds to the target analyte, and the recognition moiety of the second detection probe is binds to a different portion of the target analyte, thereby bringing the first detection probe and the second detection probe into proximity sufficient to allow for the second portion of the first detection probe to bind by complementarity to the second portion of the second detection probe, whereby the first detection probe and the second detection probe form a stable duplex,
wherein in the presence of the target analyte, the first portion of the first detection probe binds by complementarity to the overhang of the first strand of the double-stranded oligonucleotide, and the first portion of the second detection probe binds by complementarity to the internal segment of the first strand of the double-stranded oligonucleotide, whereby the binding of the first detection probe and the second detection probe to the first strand of the double-stranded oligonucleotide releases the reporter moiety from the double-stranded oligonucleotide in a), and
wherein the released reporter moiety is capable of binding by complementarity to the signaling strand of the capture probe, whereby the binding is capable of displacing the displaceable strand, thereby distancing the quencher on the displaceable strand from the quenchable detectable label on the signaling strand, allowing the detectable label to produce a detectable signal.

In some embodiments, the quenchable detectable label is a fluorophore, optionally fluorescein, rhodamine, Oregon green, eosin, Texas red, cyanine, indocarbocyanine, oxacarbocyanine, thiacarbocyanine, merocyanine, dansyl, pyridyloxazole, nitrobenzoxadiazole, benzoxadiazole, anthraquinone, cascade blue, Nile red, Nile blue, cresyl violet, oxazine 170, proflavin, acridine orange, acridine yellow, auramine, crystal violet, malachite green, porphin, phthalocyanine, bilirubin, BODIPY, aza-BODIPY 29, or a derivative thereof. In some embodiments, the quencher is [4-((4-(dimethylamino)phenyl)azo)benzoic acid] (DABCYL acid), a fluorescence resonance energy transfer (FRET), optionally a Black Hole Quencher (BHQ) or a QSY quencher, a dinitrobenzene quencher, a Qxl quencher, Iowa Black FQ, Iowa Black RQ, IRDye QC-1, or a derivative thereof.

Also provided is a use of the biosensor described herein, or a kit described herein, to determine the presence of a target analyte in a sample.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should be given the broadest interpretation consistent with the description as a whole.

### DRAWINGS

Certain embodiments of the disclosure will now be described in greater detail with reference to the attached drawings in which:

FIG. 1A shows a schematic illustration of the operating principles and the components of the bio-barcode assay in exemplary embodiments of the disclosure. FIG. 1A shows the sample is introduced into the assay vial (collect) and a drop of the sample/reagent mix (measure) is taken and placed on the electrochemical chip for measurement. Inside the assay vial, the antibody-modified DNA motifs TB and B*C bind the same protein target, inducing hybridization at a short complementary region (middle region) initiating the formation of a three way junction with the exposed region of T*C*, releasing redox-labelled CR* from the T*C*:CR* duplex through toehold mediated strand displacement.

FIG. 1B shows a schematic illustration of the operating principles and the components of the bio-barcode assay in exemplary embodiments of the disclosure. FIG. 1B shows on-chip hybridization of the redox-labelled bio-barcode: thiol binding immobilizes a partially complementary double-stranded capture probe CP:D1, followed by the addition of MCH for probe alignment and surface blocking. The released barcode displaces D1 from the CP:D1 immobilized duplex via toehold mediated strand displacement bringing the redox tag near the electrode surface, inducing an electrochemical signal.

FIG. 1C shows a schematic illustration of the operating principles and the components of the bio-barcode assay in exemplary embodiments of the disclosure. FIG. 1C shows prevention of non-specific adsorption using poly-A as a surface blocker: after immobilization of CP:D1 and MCH, poly-A is deposited and adsorbed through adenine-gold interactions, preventing non-specific adsorption of biomolecules such as proteins found in clinical samples.

FIG. 2A shows validation of the bio-barcode assay using a model streptavidin/biotin system in exemplary embodiments of the disclosure. FIG. 2A shows a schematic representation of the fluorescence bio-barcode validation assay for protein detection.

FIG. 2B shows validation of the bio-barcode assay using a model streptavidin/biotin system in exemplary embodiments of the disclosure. FIG. 2B shows generation of fluorescent signal by the release of the bio-barcode upon recognition of the streptavidin protein target, Target=10 nM streptavidin, Blank=0 nM streptavidin.

FIG. 2C shows validation of the bio-barcode assay using a model streptavidin/biotin system in exemplary embodiments of the disclosure. FIG. 2C shows validation of the e-biobarcode assay on planar gold electrodes using methylene blue as the redox reporter. Square wave voltammetry scans recorded for the increasing streptavidin concentrations from 0 nM-1000 nM with an Ag/AgCl reference electrode.

FIG. 2D shows validation of the bio-barcode assay using a model streptavidin/biotin system in exemplary embodiments of the disclosure. FIG. 2D shows the peak electrochemical current extracted from FIG. 2C in response to the increasing concentrations of streptavidin with linear trend included as an inset. The error bars represent the standard deviation from the mean. Each bar represents average data obtained from the same sample measured using at least 3 electrodes.

FIG. 3 shows native PAGE of the bio-barcode assay using 1 μM streptavidin target in an exemplary embodiment of the disclosure: lane 1 contained 1.25 nM TB, 1.25 nM B*C, 1 μM T*C*:CR; lane 2 contained 1.25 nM TB, 1.25 nM B*C, 1 μM T*C*:CR, 1 μM streptavidin; lane 3 contained 1 uM CR*; lane 4 contained 1 μM T*C*:CR*; lane 5 contained 1 μM TB; lane 6 contained 1 μM B*C.

FIG. 4 shows a schematic representation demonstrating the preparation of the biorecognition motifs in an exemplary embodiment of the disclosure.

FIG. 5A shows a graph of the results from evaluating the performance of the e-biobarcode assay for the electrochemical detection of PSA in exemplary embodiments of the disclosure. FIG. 5A shows SWV responses for increasing PSA concentrations from 0-200 ng mL⁻¹ obtained using planar electrodes with a SEM image of the planar electrode surface as an inset.

FIG. 5B shows a graph of the results from evaluating the performance of the e-biobarcode assay for the electrochemical detection of PSA in exemplary embodiments of the disclosure. FIG. 5B shows SWV responses for increasing PSA concentrations from 0-200 ng mL⁻¹ obtained using 3D nano-electrodes with a SEM image of the 3D-nano electrode surface as an inset. All electrochemical potentials are with respect to an Ag/AgCl reference electrode.

FIG. 5C shows a graph of the results from evaluating the performance of the e-biobarcode assay for the electrochemical detection of PSA in exemplary embodiments of the disclosure. FIG. 5C shows electrochemical detection of PSA on planar electrodes with peak current extracted from FIG. 5A with the linear trend in the log concentration as an inset.

FIG. 5D shows a graph of the results from evaluating the performance of the e-biobarcode assay for the electrochemical detection of PSA in exemplary embodiments of the disclosure. FIG. 5D shows electrochemical detection of PSA on 3D nano-electrodes with the peak current extracted from FIG. 5B with the linear trend in the log concentration as an inset. Each bar represents average data obtained from the same sample measured using at least 3 electrodes.

FIG. 6A shows 3D nano-electrodes for the e-biobarcode assay in exemplary embodiments of the disclosure. FIG. 6A shows a schematic of the fabrication of the 3D nano-electrodes with SEM images of the electrode surface before and after electrodeposition.

FIG. 6B shows 3D nano-electrodes for the e-biobarcode assay in exemplary embodiments of the disclosure. FIG. 6B shows a characteristic redox curve for Au in 0.5 M H₂SO₄ produced from reversible cycling from 0-1.6 V against Ag/AgCl at a scan rate of 0.1 V/s.

FIG. 7 shows fluorescent detection of PSA in reaction buffer using 25 nM TB, 25 nM B*C, 20 nM T*C*:CR*, 20 nM FAM/IOWA Black CP:D1 and 1 μg mL⁻¹ PSA in an exemplary embodiment of the disclosure.

FIG. 8A shows validation of the performance of the e-biobarcode assay in the presence of biological interfering materials in exemplary embodiments of the disclosure. FIG. 8A shows SWV responses for increasing PSA concentrations from 0-200 ng mL⁻¹ obtained using 3D nano-electrodes in undiluted human plasma. All electrochemical potentials are with respect to a Ag/AgCl reference electrode.

FIG. 8B shows validation of the performance of the e-biobarcode assay in the presence of biological interfering materials in exemplary embodiments of the disclosure. FIG. 8B shows electrochemical detection of PSA in undiluted human plasma with the linear trend of the log concentration as an inset. The peak currents are extracted from the data presented in FIG. 8A.

FIG. 8C shows validation of the performance of the e-biobarcode assay in the presence of biological interfering materials in exemplary embodiments of the disclosure. FIG. 8C shows the effect of poly-A on electrochemical signal produced in undiluted human serum using 1 ng mL⁻¹ PSA.

FIG. 8D shows validation of the performance of the e-biobarcode assay in the presence of biological interfering materials in exemplary embodiments of the disclosure. FIG. 8D shows detection of 10 ng mL⁻¹ of PSA with specific and non-specific recognition antibodies compared to blank signal obtained using anti-PSA. Each bar represents average data obtained from the same sample measured using at least 3 electrodes.

FIG. 9 shows the peak electrochemical current in response to various targets in an exemplary embodiment of the disclosure: blank solution, 10 ng mL⁻¹ IL-6, 10 ng mL⁻¹ GFAP and 10 ng mL⁻¹ PSA, all in reaction buffer. The error bars represent the standard deviation from the mean. Each bar represents average data obtained from the same sample measured using at least 3 electrodes.

### DETAILED DESCRIPTION

### I. Definitions

Unless otherwise indicated, the definitions and embodiments described in this and other sections are intended to be applicable to all embodiments and aspects of the present disclosure herein described for which they are suitable as would be understood by a person skilled in the art. It is also to be understood that the terminology used herein is for the purpose of describing particular aspects only and is not intended to be limiting.

The term “sample” or “test sample” as used herein refers to any material in which the presence or amount of a target analyte is unknown and can be determined in an assay. The sample can be from any source, for example, any biological (e.g. human or animal samples, including clinical samples), environmental (e.g. water, soil or air) or natural (e.g. plants) source, or from any manufactured or synthetic source (e.g. food or drinks). The sample can be comprised or is suspected of comprising one or more analytes. The sample can be a “biological sample” comprising cellular and non-cellular material, including, but not limited to, tissue samples, urine, blood, serum, other bodily fluids and/or secretions. The sample can be in its undiluted form or diluted in an appropriate diluent, for example, a buffer or an aqueous solution known in the art. In some embodiments, the sample comprises blood, plasma, urine, saliva, sputum, oropharyngeal and/or nasopharyngeal secretions.

The term “target”, “analyte” or “target analyte” as used herein refers to any agent, including, but not limited to, a small inorganic molecule, small organic molecule, metal ion, biomolecule, toxin, biopolymer (such as a nucleic acid, carbohydrate, lipid, peptide, protein), cell, tissue, microorganism and virus, for which one would like to sense or detect. The analyte can be either isolated from a natural source or is synthetic. The analyte can be a single compound or a class of compounds, such as a class of compounds that share structural or functional features. The term analyte also includes combinations (e.g. mixtures) of compounds or agents such as, but not limited, to combinatorial libraries and samples from an organism or a natural environment.

The term “nucleic acid” as used herein refers to a polynucleotide or oligonucleotide, such as deoxyribonucleic acid (DNA), ribonucleic acid (RNA), modified nucleotides and/or nucleotide derivatives, and can be either double-stranded (ds) or single-stranded (ss). The term “strand” as used herein is understood to refer to nucleic acid unless otherwise stated. In some embodiments, modified nucleotides can contain one or more modified bases (e.g. tritiated bases and unusual bases such as inosine), modified backbones (e.g. peptide nucleic acid, PNA) and/or other chemically, enzymatically, or metabolically modified forms.

The term “antibody” as used herein refers to a glycoprotein, or antigen-binding fragments thereof, that has specific binding affinity for an antigen as the target analyte. Antibodies can be monoclonal and/or polyclonal antibodies. Antibodies can be chimeric or humanized.

The term “detection probe” as used herein can refer to a molecule (e.g. compound) such as, but not limited to, a nucleic acid (e.g. oligonucleotide, DNAzyme, aptamer), protein (e.g. antibody, enzyme) and/or peptide that is able to recognize the presence of a target analyte (e.g. bind to the target analyte). The detection probe has a recognition moiety and a junction forming moiety. The recognition moiety is the part of the detection probe that is able to recognize the presence of a target analyte. The junction forming moiety of the detection probe is the part of the detection probe that is capable of forming a junction between two detection probes and a nucleic acid, for example, a double-stranded oligonucleotide that contains one strand (e.g. the first strand) that can interact with the detection probes, and another strand (e.g. the second strand) that is a reporter moiety. The formation of a junction between two detection probes and the first strand of the double-stranded oligonucleotide can be mediated by complementarity, for example, complementarity between a portion on the two detection probes, and complementarity between each of the probes with different portions of the first strand. This junction formation releases the strand containing the reporter moiety from the double-stranded oligonucleotide, through, for example, toehold mediated strand displacement.

The term “reporter moiety” as used herein refers to a moiety comprising a molecule (e.g. compound) for reporting the presence of an analyte. For example, the moiety is used for transducing the presence of an analyte recognized by the recognition moiety to a detectable signal. The reporter moiety can be a detectable label alone, a molecule modified with a detectable label, or a molecule without a detectable label and is able to act to distance a quenchable detectable label from its quencher in the presence of target analyte thereby facilitating the generation of a signal from the quenchable detectable label. The reporter moiety can be a molecule modified with a redox, photoelectrochemical, passivating, semi-conductive and/or conductive species.

The term “capture probe” as used herein refers to a molecule (e.g. compound) that recognizes and binds (e.g. hybridizes) to a reporter moiety. The capture probe can comprise a nucleic acid, aptamer, DNAzyme, enzyme, and/or antibody. The capture probe can be immobilized (e.g. functionalized) on a solid support, for example, on an electrode. Where the capture probe comprises a nucleic acid, it can be single-stranded or double-stranded. Where the capture probe comprises a double-stranded nucleic acid, one of the strands can be an immobilized strand attached to a solid support, or alternatively, a signaling strand that attaches or not to a solid support, and for example, comprises a quenchable detectable label or a quencher. Where the capture probe comprises a double-stranded nucleic acid, the other strand is a displaceable strand that is capable of being displaced from the double-stranded nucleic acid by another nucleic acid that has stronger complementarity to the immobilized strand or signaling strand. Where the signaling strand comprises a quenchable detectable label, the displaceable strand would comprise a suitable quencher. Where the displaceable strand comprises a quenchable detectable label, the signaling strand would comprise a suitable quencher. In some embodiments, the capture probe is immobilized or coupled to a support, for example, a solid support, for example, an electrode. In some embodiments, the capture probe comprises a biopolymer. In some embodiments, the capture probe comprises a nucleic acid having nucleic acid sequence that hybridizes to a complementary or partially complementary sequence. In some embodiments, the signaling strand comprises a quenchable detectable label and the displaceable strand comprises a quencher. In some embodiments, the signaling strand comprises a quencher and the displaceable strand comprises a quenchable detectable label.

The term “hybridization” or “hybridize” as used herein refers to the sequence specific non-covalent binding interaction with a complementary, or partially complementary, nucleic acid sequence. Binding by complementarity has the same meaning as hybridizing, referring to the sequence specific non-covalent binding interaction with a complementary, or partially complementary, nucleic acid sequence.

The term “functionalizing” or “functionalized on” as used herein refers to various common approaches for functionalizing a material, which can be classified as mechanical, physical, chemical and biological. Any suitable form of coupling can be utilized (e.g. coating, binding, etc.).

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. In addition, all ranges given herein include the end of the ranges and also any intermediate range points, whether explicitly stated or not.

As used in this disclosure, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

It will be understood that any component defined herein as being included can be explicitly excluded by way of proviso or negative limitation, such as any specific compounds or method steps, whether implicitly or explicitly defined herein.

Although methods and materials similar or equivalent to those described herein can be used in the practice or testing of this disclosure, suitable methods and materials are described below.

### II. Biosensors and Methods of the Disclosure

Combining the simplicity of a bio-barcode assay with the sensitivity of electrochemical readout offers tremendous potential for developing sensitive and specific, yet simple POC biosensors. Bio-barcode assays can be programmed to perform specific capture of molecule, for example protein, in solution, followed by the release of a short and fast-diffusing nucleic acid, for example DNA, barcode, thus eliminating the mass transport and steric hindrance issues encountered in surface-based protein biosensors. Additionally, since these assays have a built-in mechanism for releasing signal transducing probes, they can eliminate the need for the manual addition of reagents.

The electrochemical bio-barcode assay disclosed herein allows proteins to be analyzed, for example, in undiluted and unprocessed human plasma, with the analytical sensitivity and specificity that is required for clinical decision making. This analysis is performed in a sample-in-answer-out approach without the need for the sequential addition of reagents or multi-step processing, demonstrating a viable option for enabling clinical decision making at the point-of-care.

Using this assay, clinically-relevant performance for the analysis of prostate specific antigen (PSA) in undiluted and unprocessed human plasma: a log-linear range of 1 ng mL⁻¹-200 ng mL⁻¹ and a LOD of 0.4 ng mL⁻¹ is achieved. The simplicity of the e-biobarcode assay provides a solution for biomarker analysis at the point-of-care.

Accordingly, herein provided is a biosensor for detecting a target analyte in a sample comprising:
a) a double-stranded oligonucleotide comprising an overhang on a first strand of the oligonucleotide, and a second strand of the oligonucleotide that is a reporter moiety comprising a detectable label;
b) a first detection probe comprising a recognition moiety and a junction forming moiety, wherein the junction forming moiety comprises a first portion capable of binding by complementarity to the overhang of the first strand of the double-stranded oligonucleotide;
c) a second detection probe comprising a recognition moiety and a junction forming moiety, wherein the junction forming moiety comprises a first portion capable of binding by complementarity to an internal segment of the first strand of the double-stranded oligonucleotide; and
d) a capture probe functionalized on an electrode, wherein the capture probe comprises an immobilized strand attached to the electrode, and optionally a displaceable strand binding to the immobilized strand by partial complementarity;
wherein the junction forming moiety of the first detection probe comprises a second portion complementary to a second portion of the junction forming moiety of the second detection probe,
wherein in the presence of the target analyte, the recognition moiety of the first detection probe binds to the target analyte, and the recognition moiety of the second detection probe binds to a different portion of the target analyte, thereby bringing the first detection probe and the second detection probe into proximity sufficient to allow for the second portion of the first detection probe to bind by complementarity to the second portion of the second detection probe, whereby the first detection probe and the second detection probe form a stable duplex,
wherein in the presence of the target analyte, the first portion of the first detection probe binds by complementarity to the overhang of the first strand of the double-stranded oligonucleotide, and the first portion of the second detection probe binds by complementarity to the internal segment of the first strand of the double-stranded oligonucleotide, whereby the binding of the first detection probe and the second detection probe to the first strand of the double-stranded oligonucleotide releases the reporter moiety from the double-stranded oligonucleotide in a), and
wherein the released reporter moiety is capable of binding by complementarity to the immobilized strand of the capture probe attached to the electrode, whereby the binding is capable of displacing the optional displaceable strand and bringing the detectable label on the reporter moiety close to the electrode surface for producing a detectable electrochemical signal.

In some embodiments, the first strand of the oligonucleotide and the second strand of the oligonucleotide are partially complementary to one another and are capable of forming a duplex in solution in the absence of the target analyte. In some embodiments, the duplex formation is capable of preventing the first strand of the oligonucleotide from hybridizing with the first portion of the junction forming moiety of the first detection probe and preventing the first strand of the oligonucleotide from hybridizing with the first portion of the junction forming moiety of the second detection probe.

In some embodiments, the recognition moiety of the first detection probe and the recognition moiety of the second detection probe, each independently, comprises a nucleic acid, a small molecule, a peptide, or a protein. In some embodiments, the protein is an antibody or an antigen-binding fragment thereof. In some embodiments, the junction forming moiety of the first detection probe and the junction forming moiety of the second detection probe, each independently, comprises a nucleic acid. In some embodiments, the immobilized strand of the capture probe and the displaceable strand of the capture probe, each independently, comprises a nucleic acid.

The skilled person recognizes that the detectable label can be any suitable detectable label known in the art, for example, redox species or photoelectrochemical species, can be used in the biosensor described herein. In some embodiments, the detectable label comprises a redox species or photoelectrochemical species. In some embodiments, the detectable label comprises a redox species. In some embodiments, the redox species is selected from the group consisting of methylene blue, methylene blue succinimide, methylene blue maleimide, Atto MB2 maleimide, other methylene blue derivatives, 3,7-Bis-[(2-Ammoniumethyl) (methyl)amino]phenothiazin-5-ium trifluoroacetate, 3,7-Bis-(piperazin-4-ium-1-yl)phenothiazin-5-ium trifluoroacetate, 3,7-Bis-[(2-ammoniumethyl)(methyl)amino] phenothiazin-5-ium chloride, 3,7-Bis-(piperazin-4-ium-1-yl)phenothiazin-5-ium chloride, and ferrocene. In some embodiments, the redox species is methylene blue.

In some embodiments, the detectable electrochemical signal is a change in current, voltage or impedance. In some embodiments, the detectable electrochemical signal is an increase in current compared to in the absence of the target analyte. In some embodiments, the electrode comprises a conductive material, a semi-conductive material, or combinations thereof. In some embodiments, the electrode comprises a metal, a metal alloy, a metal oxide, a superconductor, a semi-conductor, a carbon-based material, a conductive polymer, or combinations thereof. In some embodiments, the electrode comprises a metal. In some embodiments, the electrode comprises gold.

The electrode can be three-dimensionally nanostructured such that electrochemical signal transduction can be functionalized with molecular layers designed to immobilize a capture probe. Three-dimensional and nanostructured transducers can enhance the efficiency of interfacial nucleic acid (e.g. DNA) strand displacement reactions and assay sensitivity. In some embodiments, the electrode comprises three-dimensional nanostructures.

The biosensor described herein can also include a surface blocker that, for example, reduces nonspecific adsorption onto an electrode surface. In some embodiments, the biosensor further comprises a surface blocker functionalized on the electrode. In some embodiments, the surface blocker comprises a poly-A oligonucleotide and/or mercaptohexanol. In some embodiments, the surface blocker comprises poly-A oligonucleotide and mercaptohexanol. In some embodiments, the biosensor further comprises a poly-A oligonucleotide functionalized on the electrode. In some embodiments, the biosensor further comprises mercaptohexanol functionalized on the electrode.

The biosensor described herein can also be a two-electrode or a three-electrode set up, that for examples, includes a counter electrode (sometimes referred to as an auxiliary electrode) and/or a reference electrode. A reference electrode refers to, for example, an electrode that has an established electrode potential. A counter electrode is, for example, an electrode that ensures that current does not pass through a reference cell, ensuring that the current is equal to that of the working electrode's current, by which the working electrode is the electrode that is attached, for example, to a capture probe. In some embodiments, the chip comprises a three-electrode set up. In some embodiments, the biosensor further comprises a counter electrode and/or a reference electrode. In some embodiments, the chip comprises a working electrode, a counter electrode and a reference electrode. In some embodiments, the working electrode comprises a metal. In some embodiments, the working electrode comprises gold. In some embodiments, the counter electrode comprises a metal. In some embodiments, the counter electrode comprises platinum. In some embodiments, the reference electrode is an Ag/AgCl reference electrode. In some embodiments, the biosensor comprises a multi-electrode electrochemical chip.

The sample that can be used in the biosensor described herein can be a sample in an undiluted state or diluted in an aqueous solution. For example, an undiluted sample be blood, plasma, urine, or saliva. In some embodiments, the sample comprises blood. In some embodiments, the sample comprises plasma. In some embodiments, the sample comprises urine. In some embodiments, the sample comprises saliva. In some embodiments, the sample is an aqueous solution.

The target analyte can be any molecule including biomolecule, for example, a small molecule, a nucleic acid, a lipid, a carbohydrate, or a protein. In some embodiments, the target analyte is a small molecule. In some embodiments, the target analyte is a biomolecule. In some embodiments, the target analyte is a nucleic acid. In some embodiments, the target analyte is a lipid. In some embodiments, the target analyte is a carbohydrate. In some embodiments, the target analyte is a protein. In some embodiments, the target analyte is prostate specific antigen.

The biosensor described herein can be used for various purposes. In some embodiments, the biosensor is for use in clinical and agricultural diagnostics, agri-food quality control, environmental monitoring, health screening, health monitoring, and/or pharmaceutical development.

In another aspect, also provided is a method of detecting a target analyte in a sample, the method comprising:
a) mixing, optionally in a solution, the sample with the double-stranded oligonucleotide, a first detection probe and a second detection probe from the biosensor described herein, to provide a mixture, wherein upon the mixing in the presence of the target analyte, a recognition moiety of the first detection probe binds to the target analyte, a recognition moiety of the second detection probe binds to a different portion of the target analyte, thereby bringing the first detection probe and the second detection probe into close proximity sufficient to allow for the second portion of the first detection probe to bind by complementarity to the second portion of the second detection probe, thereby forming a stable duplex, and wherein upon the forming of the stable duplex, the first portion of the first detection probe binds by complementarity to an overhang of the first strand of the double-stranded oligonucleotide, and the first portion of the second detection probe binds by complementarity to an internal segment of the first strand of the double-stranded oligonucleotide, thereby forming a junction and releasing a reporter moiety from the double-stranded oligonucleotide;
b) contacting the mixture with a capture probe functionalized on an electrode, wherein upon the contacting, in the presence of the target analyte, the released reporter moiety binds to an immobilized strand of the capture probe, optionally if a displaceable strand is present, displacing the displaceable strand, and bringing a detectable label on the reporter moiety close to the electrode surface, thereby producing a detectable electrochemical signal; and
c) measuring the detectable electrochemical signal produced from the electrode.

In some embodiments, the detectable electrochemical signal is a change in current, voltage or impedance in the presence of the target analyte compared to in the absence of the target analyte. In some embodiments, the detectable electrochemical signal is an increase in current compared to in the absence of the target analyte. In some embodiments, the target analyte is a protein. In some embodiments, the protein is prostate specific antigen. In some embodiments, the method comprises a single step operation. In some embodiments, the method comprises sample-in-answer-out (SIAO) operation. In some embodiments, the target analyte is a biomolecule. In some embodiments, the target analyte is a nucleic acid. In some embodiments, the target analyte is a protein. In some embodiments, the target analyte is prostate specific antigen.

In another aspect, also provided is a system comprising the biosensor described herein, and an ammeter, a voltameter, or an impedance analyzer.

In another aspect, also provided is a kit for detecting a target analyte in a sample, wherein the kit comprises:
a) a double-stranded oligonucleotide comprising an overhang on a first strand of the oligonucleotide, and a second strand of the oligonucleotide that is a reporter moiety comprising a detectable label;
b) a first detection probe comprising a recognition moiety and a junction forming moiety, wherein the junction forming moiety comprises a first portion capable of binding by complementarity to the overhang of the first strand of the double-stranded oligonucleotide;
c) a second detection probe comprising a recognition moiety and a junction forming moiety, wherein the junction forming moiety comprises a first portion capable of binding by complementarity to an internal segment of the first strand of the double-stranded oligonucleotide; and
d) instructions for use.

In some embodiments, the kit further comprises a capture probe functionalized on an electrode, wherein the capture probe comprises an immobilized strand attached to the electrode, and optionally a displaceable strand binding to the immobilized strand by partial complementarity, and optionally further comprising at least one of a solution, a sample collector, a liquid dropper, a lancet, a bandage, gloves, and a mask. In some embodiments, the target analyte is a target analyte described herein. In some embodiments, the sample is a sample described herein.

In another aspect, also provided is a kit for detecting a target analyte in a sample, wherein the kit comprises components required for a method described herein and instructions for use of the kit.

In another aspect, also provided is a biosensor for detecting a target analyte in a sample comprising:
a) a double-stranded oligonucleotide comprising an overhang on a first strand of the oligonucleotide, and a second strand of the oligonucleotide that is a reporter moiety;
b) a first detection probe comprising a recognition moiety and a junction forming moiety, wherein the junction forming moiety comprises a first portion capable of binding by complementarity to the overhang of the first strand of the double-stranded oligonucleotide;
c) a second detection probe comprising a recognition moiety and a junction forming moiety, wherein the junction forming moiety comprises a first portion capable of binding by complementarity to an internal segment of the first strand of the double-stranded oligonucleotide; and
d) a capture probe, optionally functionalized on a solid support, wherein the capture probe comprises a signaling strand and a displaceable strand binding to the signaling strand by partial complementarity, wherein the signaling strand comprises a quenchable detectable label and the displaceable strand comprises a quencher in sufficiently close proximity to and capable of quenching the quenchable detectable label;
wherein the junction forming moiety of the first detection probe comprises a second portion complementary to a second portion of the junction forming moiety of the second detection probe,
wherein in the presence of the target analyte, the recognition moiety of the first detection probe binds to the target analyte, and the recognition moiety of the second detection probe binds to a different portion of the target analyte, thereby bringing the first detection probe and the second detection probe into proximity sufficient to allow for the second portion of the first detection probe to bind by complementarity to the second portion of the second detection probe, whereby the first detection probe and the second detection probe form a stable duplex,
wherein in the presence of the target analyte, the first portion of the first detection probe binds by complementarity to the overhang of the first strand of the double-stranded oligonucleotide, and the first portion of the second detection probe binds by complementarity to the internal segment of the first strand of the double-stranded oligonucleotide, whereby the binding of the first detection probe and the second detection probe to the first strand of the double-stranded oligonucleotide releases the reporter moiety from the double-stranded oligonucleotide in a), and
wherein the released reporter moiety is capable of binding by complementarity to the signaling strand of the capture probe, whereby the binding is capable of displacing the displaceable strand, thereby distancing the quencher on the displaceable strand from the quenchable detectable label on the signaling strand, allowing the detectable label to produce a detectable signal.

In some embodiments, the first strand of the oligonucleotide and the second strand of the oligonucleotide are partially complementary to one another and are capable of forming a duplex in solution in the absence of the target analyte. In some embodiments, the duplex formation is capable of preventing the first strand of the oligonucleotide from hybridizing with the first portion of the junction forming moiety of the first detection probe and preventing the first strand of the oligonucleotide from hybridizing with the first portion of the junction forming moiety of the second detection probe.

In some embodiments, the recognition moiety of the first detection probe and the recognition moiety of the second detection probe, each independently, comprises a nucleic acid, a small molecule, a peptide, or a protein. In some embodiments, the protein is an antibody or an antigen-binding fragment thereof. In some embodiments, the junction forming moiety of the first detection probe and the junction forming moiety of the second detection probe, each independently, comprises a nucleic acid. In some embodiments, the signaling strand of the capture probe and the displaceable strand of the capture probe, each independently, comprises a nucleic acid.

The skilled person recognizes the quenchable detectable label can be any suitable quenchable detectable label known in the art, for example, a fluorophore, and the corresponding quencher can be any compatible quencher known in the art. In some embodiments, the quenchable detectable label is a fluorophore, optionally fluorescein, rhodamine, Oregon green, eosin, Texas red, cyanine, indocarbocyanine, oxacarbocyanine, thiacarbocyanine, merocyanine, dansyl, pyridyloxazole, nitrobenzoxadiazole, benzoxadiazole, anthraquinone, cascade blue, Nile red, Nile blue, cresyl violet, oxazine 170, proflavin, acridine orange, acridine yellow, auramine, crystal violet, malachite green, porphin, phthalocyanine, bilirubin, BODIPY, aza-BODIPY 29, or a derivative thereof. In some embodiments, the quencher is [4-((4-(dimethylamino)phenyl)azo)benzoic acid] (DABCYL acid), a fluorescence resonance energy transfer (FRET), optionally a Black Hole Quencher (BHQ) or a QSY quencher, a dinitrobenzene quencher, a Qxl quencher, Iowa Black FQ, Iowa Black RQ, IRDye QC-1, or a derivative thereof.

In another aspect, also provided is a method of detecting a target analyte in a sample, the method comprising:
a) mixing, optionally in a solution, the sample with the double-stranded oligonucleotide, a first detection probe and a second detection probe from the biosensor described herein, to provide a mixture, wherein upon the mixing in the presence of the target analyte, the recognition moiety of the first detection probe binds to the target analyte, the recognition moiety of the second detection probe binds to a different portion of the target analyte, thereby bringing the first detection probe and the second detection probe into close proximity sufficient to allow for the second portion of the first detection probe to bind by complementarity to the second portion of the second detection probe, thereby forming a stable duplex, and wherein upon the forming of the stable duplex, the first portion of the first detection probe binds by complementarity to an overhang of the first strand of the double-stranded oligonucleotide, and the first portion of the second detection probe binds by complementarity to an internal segment of the first strand of the double-stranded oligonucleotide, thereby forming a junction and releasing the reporter moiety from the double-stranded oligonucleotide;
b) in the presence of the target analyte, the released reporter moiety binds to a signaling strand of a capture probe, wherein the signaling strand comprises a quenchable detectable label, the binding thereby displaces a displaceable strand comprising a quencher from the capture probe, thereby producing a detectable signal; and
c) measuring the detectable signal.

In some embodiments, the target analyte is a protein. In some embodiments, the protein is prostate specific antigen. In some embodiments, the method comprises a single step operation. In some embodiments, the method comprises sample-in-answer-out (SIAO) operation. In some embodiments, the target analyte is a biomolecule. In some embodiments, the target analyte is a nucleic acid. In some embodiments, the target analyte is a protein. In some embodiments, the target analyte is prostate specific antigen. In some embodiments, the quenchable detectable label is a quenchable detectable label described herein. In some embodiments, the quencher is a quencher described herein.

In another aspect, also provided is a kit for detecting a target analyte in a sample, wherein the kit comprises:
a) a double-stranded oligonucleotide comprising an overhang on a first strand of the oligonucleotide, and a second strand of the oligonucleotide that is a reporter moiety;
b) a first detection probe comprising a recognition moiety and a junction forming moiety, wherein the junction forming moiety comprises a first portion capable of binding by complementarity to the overhang of the first strand of the double-stranded oligonucleotide;
c) a second detection probe comprising a recognition moiety and a junction forming moiety, wherein the junction forming moiety comprises a first portion capable of binding by complementarity to an internal segment of the first strand of the double-stranded oligonucleotide; and
d) instructions for use.

In some embodiments, the kit further comprises a capture probe, wherein the capture probe comprises a signaling strand comprising a quenchable detectable label, and a displaceable strand comprising a quencher, wherein the displaceable strand binds to the signaling strand by partial complementarity, and optionally further comprising at least one of a solution, a sample collector, a liquid dropper, a lancet, a bandage, gloves, and a mask. In some embodiments, the quenchable detectable label is a quenchable detectable label described herein. In some embodiments, the quencher is a quencher described herein. In some embodiments, the target analyte is a target analyte described herein. In some embodiments, the sample is a sample described herein.

Also provided is a use of the biosensor described herein, or a kit described herein, to determine the presence of a target analyte in a sample.

In another aspect, also provided is a biosensor for detecting a target analyte in a sample comprising two detection probes; a first single-stranded oligonucleotide partially complementary to a segment of each of the two detection probes; a second single-stranded oligonucleotide; a reporter moiety comprising a detectable label; an electrode; and a capture probe functionalized on the electrode, wherein binding of the two detection probes to the target analyte in a sample results in release of the reporter moiety to produce a detectable electrochemical signal.

In some embodiments, the two detection probes bind to the target analyte in solution. In some embodiments, binding to the target analyte induces the two detection probes to come into close proximity to one another. In some embodiments, the detection probe comprises a nucleic acid and/or an antibody. In some embodiments, the detection probe comprises a nucleic acid functionalized to an antibody. In some embodiments, the sample is an aqueous solution.

In some embodiments, the first single-stranded oligonucleotide preferentially hybridizes to the two detection probes in the presence of the target analyte. In some embodiments, the first single-stranded oligonucleotide preferentially hybridizes to the two detection probes in the presence of the target analyte in solution. In some embodiments, the reporter moiety comprises a nucleic acid. In some embodiments, the capture probe comprises a nucleic acid. In some embodiments, the first single-stranded oligonucleotide hybridizes to the reporter moiety and the second single-stranded oligonucleotide hybridizes to the capture probe in the absence of the target analyte. In some embodiments, the first single-stranded oligonucleotide and the reporter moiety are partially complementary to one another and form a duplex in solution in the absence of the target analyte. In some embodiments, the duplex formation prevents the first single-stranded oligonucleotide from hybridizing with the two detection probes in the absence of the target analyte. In some embodiments, the second single-stranded oligonucleotide and the capture probe are partially complementary to one another and form a duplex in on the electrode surface in the absence of the target analyte.

In some embodiments, the reporter moiety preferentially hybridizes to the capture probe in the presence of the target analyte. In some embodiments, binding of the two detection probes to the target analyte in a sample releases the reporter moiety from the first single-stranded oligonucleotide. In some embodiments, binding of the two detection probes to the target analyte in a sample releases the reporter moiety from the first single-stranded oligonucleotide in solution. In some embodiments, the reporter moiety that is released hybridizes to the capture probe on the electrode surface in the presence of the target analyte.

In some embodiments, the first single-stranded oligonucleotide hybridizes to the second single-stranded oligonucleotide and the reporter moiety hybridizes to the capture probe in the absence of the target analyte. In some embodiments, the first single-stranded oligonucleotide and the second single-stranded oligonucleotide are partially complementary to one another and form a duplex in solution in the absence of the target analyte. In some embodiments, the duplex formation prevents the first single-stranded oligonucleotide from hybridizing with the two detection probes in the absence of the target analyte. In some embodiments, the reporter moiety and the capture probe are partially complementary to one another and form a duplex in on the electrode surface in the absence of the target analyte.

In some embodiments, the reporter moiety preferentially hybridizes to the second single-stranded oligonucleotide in the presence of the target analyte. In some embodiments, binding of the two detection probes to the target analyte in a sample releases the second single-stranded oligonucleotide from the first single-stranded oligonucleotide. In some embodiments, binding of the two detection probes to the target analyte in a sample releases the reporter moiety from the capture probe. In some embodiments, binding of the two detection probes to the target analyte in a sample releases the second single-stranded oligonucleotide from the first single-stranded oligonucleotide in solution. In some embodiments, the preferentially hybridization of the reporter moiety to the second single-stranded oligonucleotide releases the reporter moiety from the capture probe on the electrode surface in the presence of the target analyte.

In some embodiments, the detectable electrochemical signal is a change in current, voltage or impedance. In some embodiments, the detectable electrochemical signal is an increase in current. In some embodiments, the detectable electrochemical signal is an increase in current compared to in the absence of the target analyte. In some embodiments, the detectable label comprises a redox or photoelectrochemical species. In some embodiments, the detection label comprises a redox species. In some embodiments, the redox species is selected from methylene blue, methylene blue succinimide, methylene blue maleimide, Atto MB2 maleimide (Sigma Aldrich), other methylene blue derivatives, 3,7-Bis-[(2-Ammoniumethyl) (methyl)amino]phenothiazin-5-ium trifluoroacetate, 3,7-Bis-(piperazin-4-ium-1-yl)phenothiazin-5-ium trifluoroacetate, 3,7-Bis-[(2-ammoniumethyl)(methyl)amino] phenothiazin-5-ium chloride, 3,7-Bis-(piperazin-4-ium-1-yl)phenothiazin-5-ium chloride, and ferrocene. In some embodiments, the redox species is methylene blue.

In some embodiments, the electrode comprises conductive materials, semi-conductive materials, or combinations thereof. In some embodiments, the electrode comprises metals, metal alloys, metal oxides, superconductors, semi-conductors, carbon-based materials, conductive polymers, or combinations thereof. In some embodiments, the electrode comprises metals. In some embodiments, the electrode comprises gold.

In some embodiments, the electrode comprises three-dimensional nanostructures. In some embodiments, the biosensor further comprises a poly-A oligonucleotide functionalized on the electrode. In some embodiments, the biosensor further comprises mercaptohexanol functionalized on the electrode. In some embodiments, the biosensor further comprises a counter electrode and/or a reference electrode. In some embodiments, the biosensor comprises a multi-electrode electrochemical chip. In some embodiments, the chip comprises a three-electrode set up. In some embodiments, the chip comprises a working electrode, a counter electrode and a reference electrode.

In some embodiments, the target analyte is a biomolecule. In some embodiments, the target analyte is a nucleic acid. In some embodiments, the target analyte is a protein. In some embodiments, the target analyte is prostate specific antigen.

In some embodiments, the biosensor is used for clinical and agricultural diagnostics, agri-food quality control, environmental monitoring, health screening, health monitoring, and/or pharmaceutical development.

Also provided herein is a device comprising the biosensor disclosed herein.

Also provided herein is a method of detecting a target analyte in a sample, the method comprising:
mixing two detection probes and a first single-stranded oligonucleotide, optionally hybridized to a reporter moiety or a second single-stranded oligonucleotide, disclosed herein with a sample suspected of comprising the target analyte; contacting the mixture with an electrode disclosed herein comprising a capture probe, optionally hybridized to the second single-stranded oligonucleotide or the reporter moiety; and measuring a detectable electrochemical signal from the electrode.

In some embodiments, the detectable electrochemical signal is a change in current, voltage or impedance. In some embodiments, the detectable electrochemical signal is an increase in current compared to in the absence of the target analyte.

In some embodiments, the method comprises a single step operation. In some embodiments, the method comprises sample-in-answer-out (SIAO) operation.

In some embodiments, the sample is an aqueous solution.

In some embodiments, the target analyte is a biomolecule. In some embodiments, the target analyte is a nucleic acid. In some embodiments, the target analyte is a protein. In some embodiments, the target analyte is prostate specific antigen.

Also provided herein is a kit for detecting a target analyte in a sample, wherein the kit comprises the biosensor and/or components required for the method disclosed herein and instructions for use of the kit.

Also provided herein is use of the biosensor, device and/or kit disclosed herein to determine the presence of a target analyte in a sample.

### Examples

The following non-limiting examples are illustrative of the present disclosure:

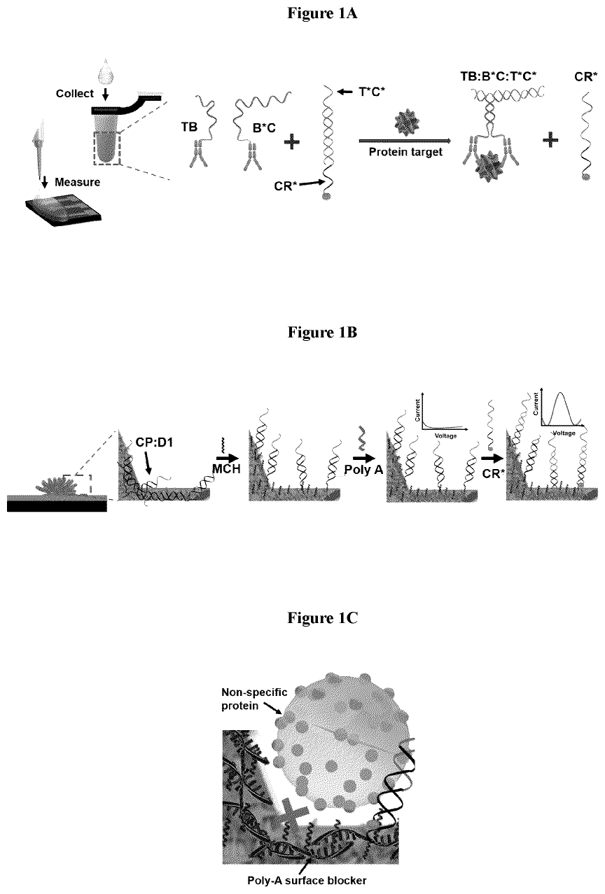

To create a SIAO electrochemical bio-barcode assay (e-biobarcode assay) for analyzing clinical samples, three components were integrated: (1) a proximity-induced bio-barcode assay, designed for electrochemical signal transduction using one-pot operation, with (2) electrochemical readout using three dimensional nanostructured electrodes, optimized for enhanced sensitivity, and (3) a surface coating of poly adenine (poly-A), used for reducing non-specific adsorption and biofouling (FIG. 1).

### Materials and Methods

The list below presents the DNA sequences used in this disclosure, written for the 5′-3′:

TB:
(32 nts; SEQ ID NO: 1)
/5BioTinTEG/TTTTTTTTTTTTTTTGTGAGGTTCGTGTGATG
B*C:
(41 nts; SEQ ID NO: 2)
AAGCGTGTATCCCATGTGTCCCTCACTTTTTTTTTTTTTTT/3BioTEG/
T*C*:
(29 nts; SEQ ID NO: 3)
CATCACACGGACACATGGGATACACGCTT
CR*:
(38 nts; SEQ ID NO: 4)
MB-TCTTCCAATCAGTCTCTCAAGCGTGTATCCCATGTGTC
D1:
(20 nts, SEQ ID NO: 5)
TCTTCCAATCAGTCTCTCAA
CP:
(27 nts, SEQ ID NO: 6)
TACACGCTTGAGAGACTGATTGGAAGA/3ThioMC3-D/
Poly-A:
(22 nts, SEQ ID NO: 7)
AAAAAAAAAAAAAAAAAAAAAA
FAM-CP:
(27 nts, SEQ ID NO: 8)
TACACGCTTGAGAGACTGATTGGAAGA/36-FAM/
IOWA Black-D1:
(20 nts, SEQ ID NO: 9)
/5IABkFQ/TCTTCCAATCAGTCTCTCAA

Materials: Magnesium chloride (MgCl₂, ≥99.0%), sodium chloride (NaCl, ≥99.0%), phosphate buffer solution (1.0 M, pH 7.4), 6-mercapto-1-hexanol (MCH, 99%), tris(2-carboxyethyl)phosphine hydrochloride (TCEP), potassium hexacyanoferrate(II) trihydrate ([Fe(CN)₆]^{4-/3-}, ≥99.95%), gold(III) chloride solution (HAuCl₄, 99.99%), 100× tris-EDTA (TE, pH 7.4), 10× tris borate-ETDA (TBE, pH 8.3), Tween 20, bovine serum albumin (BSA), streptavidin from Streptomyces avidinii, biotin, prostate-specific antigen from human semen (PSA), glial fibrillary acidic protein from human brain (GFAP), were purchased from Sigma-Aldrich (Oakville, Canada). Biotinylated human kallikrein 3/PSA polyclonal antibody (goat IgG) and biotinylated normal goat IgG control was purchased from R&D Systems (Minneapolis, MN). SYBR gold nucleic acid gel stain, DNA gel loading dye (6×), acrylamide solution (40%), ammonium persulfate (APS), 10× sterile phosphate buffer saline (PBS, pH 7.4) and tetramethylethylenediamine (TEMED) were purchased from Thermo Fisher Scientific (Mississauga, Canada). Sulfuric acid (H₂SO₄, 98%) and 2-propanol (99.5%) were purchased from Caledon Laboratories (Georgetown, Canada). Ethanol was purchased from Commercial Alcohols (Brampton, Canada). Hydrochloric acid (37% w/w) was purchased from LabChem (Zelienople, PA). Human plasma was donated by the Canadian Plasma Resources (Saskatoon, Canada). All reagents were of analytical grade and were used without further purification. Milli-Q grade ultrapure water (18.2 MΩ·cm) was used to prepare all solutions and for all washing steps. Methylene blue modified sequences were purchased from Biosearch Technologies (Novato, CA) and purified by dual high-performance liquid chromatography (HPLC). All other DNA samples were purchased from Integrated DNA Technologies (Coralville, IA) and purified by HPLC.

Duplex preparation for the bio-barcode assay: The barcode containing duplex T*C*:CR* was prepared at a final concentration of 10 μM by mixing 10 μL of T*C* and 12 μL of CR*, each at an initial concentration of 50 μM, in 28 μL of annealing buffer (1×TE, 10 mM MgCl₂, 0.05% Tween20). The mixture was heated to 90° C. for 5 minutes and then the solution was brought to 25° C. incrementally over 30 minutes. The fluorescent capture beacon was prepared at a final concentration of 10 μM by mixing 10 μL of FAM labelled CP with 15 μL of IOWA Black labelled D1, each at an initial concentration of 50 μM in 25 μL of annealing buffer. The mixture was heated to 90° C. for 5 minutes and then the solution was brought to 25° C. incrementally over 30 minutes. The same procedure was followed for the thiolated capture probe used for all electrochemical detection; 10 μL of CP was mixed with 15 μL D1, each at an initial concentration of 50 μM, in 25 ptL annealing buffer. The mixture was heated to 90° C. for 5 minutes and then the solution was brought to 25° C. incrementally over 30 minutes.

Recognition probe preparation: DNA probes for detection of PSA were prepared following a previously published protocol by Li et al.^{[1]} Briefly, 25 μL of 2.5 μM TB or B*C was mixed with 25 μL of 3 μM streptavidin (both diluted in 1×PBS containing 0.01% BSA) and incubated at 37° C. for 30 minutes, followed by 30 minutes at 25° C. A 50 μL solution of biotinylated anti-PSA prepared in 1×PBS was added to the mixture and incubated for 1 hour at 25° C. followed by 2 hours at 4° C. The recognition probes were then diluted with 150 μL of biotin solution (1×TE, 1 mM biotin, 0.01% BSA) to 250 nM and left at 4° C. overnight.

Preparation of the sensing surfaces: Pre-stressed polystyrene substrates (Graphix Shrink Film, Maple Heights, OH) were cleaned with ethanol, DI water, and then dried with air. Following the solvent cleaning step, a vinyl mask (BDF Graphics, Toronto, Canada) was put on the PS substrate and the electrode design was cut into the mask using a CraftRobo Pro (Graphtec, Tokyo, Japan). Afterwards, a gold layer was sputtered onto the surface using a Torr (DC/RF) physical vapour deposition system. The gold electrodes were then prepared for probe deposition by first electrochemically cleaning by running reversible cyclic voltammetry (CV) scans in 0.5 M H₂SO₄ from 0-1.6 V at a scan rate of 0.1 V/s until the reduction peak was stable. The electrodes were then held at a high potential of 1 V For 10 seconds, followed by a low potential of −1 V for 10 seconds. The pre-annealed thiol modified probe (CP:D1) was reduced at a final concentration of 500 nM using a 50 mM TCEP solution in deposition buffer (25 mM phosphate buffer solution, 25 mM NaCl, 100 mM MgCl₂) for 2 hours in the dark at room temperature. After reduction, 3 μL of reduced probe solution was dropped on the surface of the clean working electrode and left in the dark at room temperature for 16 hours. Non-specifically adsorbed probe was then washed using wash buffer (25 mM phosphate buffer solution, 25 mM NaCl) and a CV scan was performed from 0-0.5 V in 2 mM [Fe(CN)₆]^{4-/3-} to ensure immobilization. Measuring the CV curves of electrodes in [Fe(CN)₆]^{4-/3-} can be used to qualitatively determine the surface passivation of the electrodes by assessing the oxidation and reduction peaks. On bare gold, [Fe(CN)₆]⁴⁻ can access the surface of the electrode and can be easily oxidized to [Fe(CN)₆]³⁻ followed by reduction back to [Fe(CN)₆]⁴⁻, producing the characteristic redox curve. When negatively charged DNA is deposited on the surface of the electrodes the anions are repelled hindering the redox activity of the species, resulting in a flat curve with no peaks. MCH is used to both remove the non-specifically adsorbed DNA by competing for free gold sites and aligning the DNA probe by filling the self-assembled monolayer and slightly repelling DNA with the hydroxide. Therefore, an MCH backfill step was done using 100 mM MCH for 20 minutes, followed by another CV scan in 2 mM [Fe(CN)₆]^{4-/3-} to ensure both removal of non-specifically adsorbed probe, and aligning of specifically adsorbed probe, with washing between each step. A 3 μL solution of 1 μM poly-A was deposited onto the surface of the electrode for 30 minutes at room temperature. The drop was removed using a KimWipe, but the electrode was not washed. The electrode was then ready for electrochemical detection experiments. All electrochemical experiments were carried out on a CHI 420b with a three-electrode set-up with a gold electrode as the working electrode, an Ag/AgCl as the reference and a platinum wire as the counter electrode.

Fluorescence validation assay: For verification that a signal could be induced through recognition of the released bio-barcode, 10 μL of 250 nM TB and B*C were mixed with 10 μL of 100 nM streptavidin, and 60 μL reaction buffer (1×PBS, 10 mM MgCl₂, 0.05% Tween 20) and incubated at 37° C. for 30 minutes. A blank solution was prepared by adding 10 μL of buffer in place of streptavidin. 10 μL of 200 nM T*C*:CR* was added to the solution and directly after mixing, an 81 μL volume was put into a well of a 96-well plate. A 9 μL solution of the FAM/IOWA Black labelled CP:D1 capture beacon was added to the well and fluorescence was immediately measured every minute for 60 minutes. All experiments were done in duplicates.

Verification of complex DNA structure formation using native PAGE: All sequences were prepared at a concentration of 1 μM with reaction buffer. For target analysis, a reaction mixture containing 1.25 μM TB, 1.25 μM B*C, 1 μM T*C*:CR* and 1 μM streptavidin was prepared with reaction buffer. For the blank analysis, reaction buffer was used in place of streptavidin. The reaction mixture was incubated for 30 minutes at 37° C. and then all samples were mixed in a 5:1 ratio with loading dye and loaded into a freshly prepared 10% gel. A voltage of 80 mV was applied to the gel until separation was achieved. After separation, the gel was submerged in a 10000× dilute SYBR Gold solution for 40 minutes and then imaged on a Chemidoc MP.

Electrochemical validation assay: To verify that protein detection could be performed using electrochemical analysis, 10 μL of 250 nM TB and B*C were mixed with 10 μL of varying streptavidin concentrations and 60 μL of reaction buffer. A blank solution was prepared by adding 10 μL of reaction buffer in place of streptavidin. The solution was incubated at 37° C. for 30 minutes followed by the addition of 10 μL of 200 nM T*C*:CR*. After mixing, 3 μL of the reaction solution was deposited onto the prepared sensing electrode and the electrode was placed in a humidity chamber for incubation at 37° C. for 45 minutes. The electrode was then washed with washing buffer and SWV was performed from 0-(−0.5) V in washing buffer.

The LOD was calculated using the linear regression equation of the most linear region and the limit-of-blank (LOB). LOB is defined as the highest signal (also known as the upper prediction limit) obtained in response to a solution that is void of target analyte and is calculated by the equation LOB=mean of blank+3x (standard deviation of the blank). The LOB value was then substituted in the regression line equation to obtain the value of “x” which denotes the minimum concentration that can be reliably distinguished from the analytical noise (blank signal). This method of LOD calculation was done to take into consideration the peak current that is produced via non-specific interactions within a blank solution and was used for all subsequent protein quantification. All experimental data points obtained for each concentration including blank were measured in triplicates.

Electrochemical quantification of PSA: For the detection of PSA in PBS and undiluted human plasma, 10 μL of 250 nM antibody conjugated TB and B*C were mixed with 10 μL of varying PSA concentrations and 60 μL of either the reaction buffer or undiluted plasma. A blank solution was also prepared by adding 10 μL of either reaction buffer or undiluted plasma in place of PSA. The solution was incubated at 37° C. for 30 minutes then 10 μL of 200 nM T*C*:CR* duplex was added and the solution was mixed. Directly after mixing, 3 μL of the solution was deposited onto the prepared sensing electrode and the electrode was then placed in a humidity chamber and incubated at 37° C. for 45 minutes. The electrodes were then washed in washing buffer and SWV was performed from 0-(−0.5) V in washing buffer. All experiments were done in triplicate.

Nanostructuring of the planar electrodes: A 10 mM HAuCl₄ solution was prepared by mixing 30 mL of 0.5 M HCl with 207.9 μL of stock HAuCl₄, followed by degassing with nitrogen for 20 minutes. Planar electrodes were cleaned by rinsing in isopropanol and DI water. The clean planar electrodes were then held at a potential of −0.7 V for 600 seconds in the degassed 10 mM HAuCl₄ solution. The electrodes were then rinsed with DI water and stored for later used at room temperature.

Determining specificity of bio-barcode assay: For antibody specificity experiments, control recognition probes (TB and B*C) that were not specific for PSA biorecognition were prepared following the previous protocol (for “electrochemical validation assay”) using normal goat IgG control in place of anti-PSA. Electrochemical protein detection was done using 10 μL of 250 nM control TB, 10 μL of 250 nM control B*C, 10 μL of 200 nM T*C*:CR*, 10 μL of 10 ng mL⁻¹ PSA and 60 μL of reaction buffer.

Studying the effect of poly-A on the generated electrochemical signal: To investigate the effect of poly-A on signal generation, the same protocol was followed for preparation of the sensing surface, except deposition of poly-A was omitted. The same steps for electrochemical PSA detection using anti-PSA conjugated recognition probes were followed using 10 μL of 250 nM TB, 10 μL of 250 nM B*C, 10 μL of 200 nM T*C*:CR*, 10 μL of 100 ng mL⁻¹ IL-6 or 10 μL of 100 ng mL⁻¹ GFAP and 60 μL of reaction buffer. GFAP and IL-6 were prepared using 1×PBS.

Electrochemical Experiments: All electrochemical experiments were performed on a CHI 420b using a three-electrode set up with an Au working electrode, an Ag/AgCl reference electrode and a platinum wire counter electrode. Detection experiments were performed using SWV scanning from 0-(−0.5) V with a step potential of 0.001 V, an amplitude of 0.025 V and a frequency of 60 Hz.

### Results and Discussion

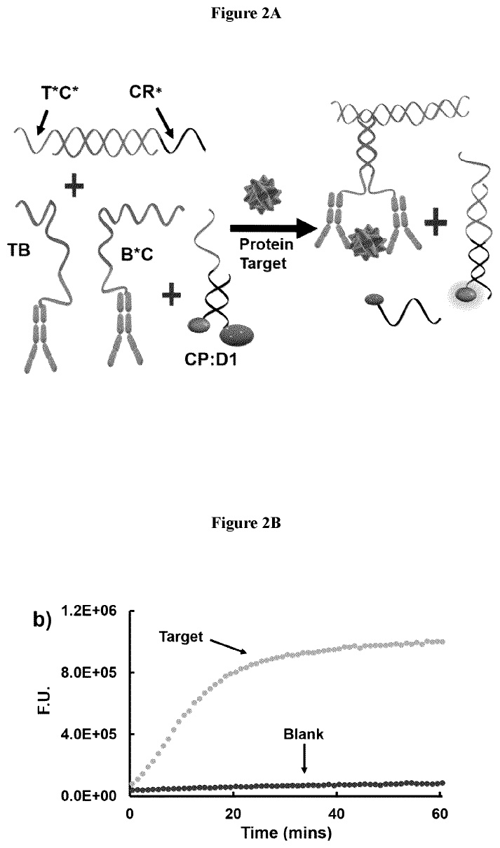

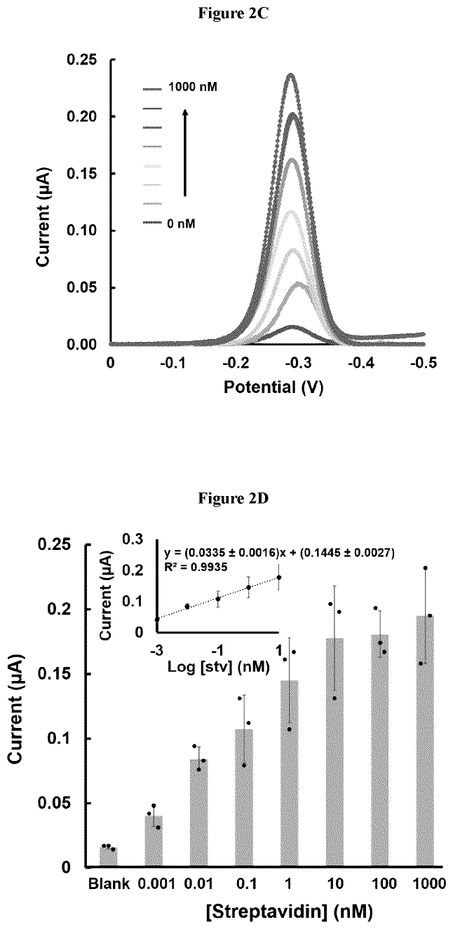

In order to validate the designed bio-barcode method, it was first verified that protein binding triggers the release of a barcode DNA strand via a real-time fluorescence assay, using streptavidin as a model protein target. Streptavidin was captured using the biotin molecules (K_{d}=10⁻¹⁵) modified at the 5′- and 3′-end of TB and B*C DNA motifs, respectively. TB and B*C were specifically designed to contain a six base pair long complementary region (middle domain in FIG. 2A) that does not form a stable duplex at room temperature. Recognition of the same streptavidin molecule by both sequences forces them to come closer together, via a poly-thyamine spacer (FIG. 1A, bottom domain) that has been previously optimized to be compatible with protein targets of various sizes, hybridize, and form a DNA/protein complex (FIG. 2A). Hybridization of the toehold region in T*C* (terminal domain) to the exposed single stranded regions of the TB:B*C duplex allows for strand displacement, resulting in the release of CR*. In the following signal generation step, a fluorophore(CP)- and quencher(D1)-labeled partial DNA duplex probe (CP:D1) was utilized, in which the released reporter CR* displaces the quencher strand, generating a fluorescence signal. Using this assay design, the fluorescent signal was measured in the presence and absence of the target analyte. At 20 minutes, a signal-to-blank ratio of 13.5 was measured (FIG. 2B), indicating a robust and rapid assay with little interference from unbound biorecognition elements.

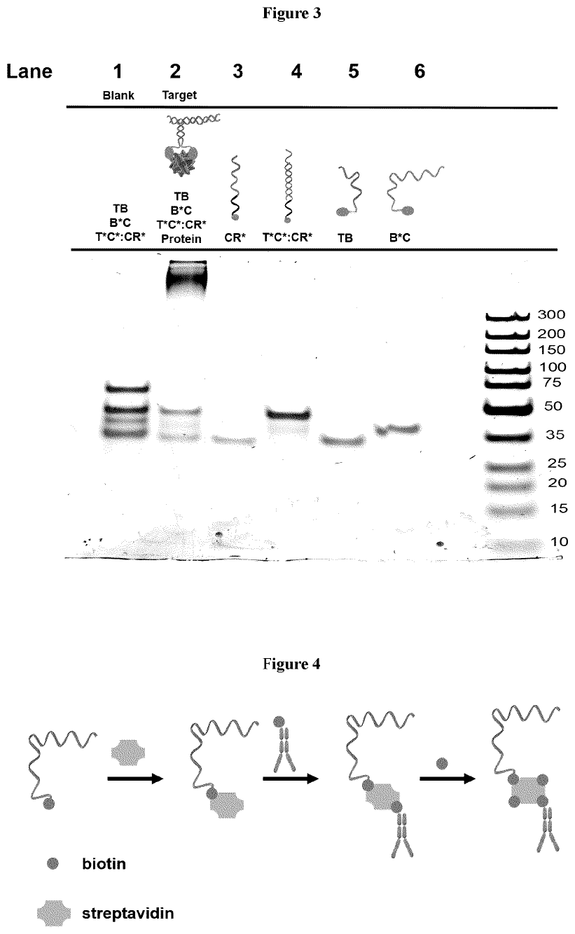

The successful formation of the designed DNA structure and the release of the barcode were further verified with native polyacrylamide gel electrophoresis PAGE (FIG. 3), using biotinylated TB and B*C strands as the biorecognition element with 1 μM of streptavidin as the protein target to demonstrate protein capture and bio-barcode release. Lane 1 contained the blank which had 1.25 nM TB, 1.25 nM B*C and 1 μM T*C*:CR; lane 2 contained the target solution with 1.25 nM TB, 1.25 nM B*C, 1 μM T*C*:CR and 1 μM streptavidin; lane 3 contained 1 μM CR*; lane 4 contained 1 μM T*C*:CR*; lane 5 contained 1 μM TB; lane 6 contained 1 μM B*C. The formation of the TWJ can be seen as a wide band with the least migration distance, only forming in lane 2 with the presence of the protein target. Additionally, a less prominent T*C*:CR* band and an increase in the CR* band can also be seen in lane 2, showing the formation of the TWJ leads to the release of the bio-barcode. Alternatively, when there is no protein target present (lane 1) the TWJ does not form and there is a prominent T*C*:CR band, indicating that without protein target the bio-barcode cannot be released.

After demonstrating that the bio-barcode assay is capable of generating the designed products using fluorescence, it was integrated with electrochemical readout. This electrochemical assay performs protein capture in solution, followed by on-chip hybridization of the released barcode at the electrode surface. This design enables an important step of protein capture to occur in solution, circumventing the diffusion and steric hindrance limitations that are encountered in surface-based antibody/protein binding.

The assay was re-engineered for electrochemical readout by immobilizing the capture probe (CP:D1 complex) on the electrode surface, eliminating the quencher and fluorophore needed in the fluorescent assay, and modifying the CR* strand with an electrochemical reporter (methylene blue (MB)). The 3D nanostructured gold electrode used for electrochemical signal transduction was functionalized with three molecular layers designed to capture the desired target (capture probe, CP:D1) and repel the biological background (mercaptohexanol (MCH) and poly-A). The result was an SIAO system where the sample was introduced into a vial containing the reaction mix, followed by adding a drop of that solution to the chip, where the electrochemical measurement was performed (FIG. 1A). In the presence of the target analyte, the released barcode CR* is designed to hybridize with the immobilized capture probe and displace D1, which brings the MB moiety close to the electrode surface, thus generating an electrochemical signal (FIG. 1A and FIG. 1B). To examine the feasibility of the e-biobarcode assay, a baseline system was established using streptavidin as the target and planar gold as the electrochemical transducer. Increasing the protein concentration increased the current generated by the reduction of MB, demonstrating a signal-on electrochemical sensor (FIG. 2C). This sensor demonstrates a log-linear response in the 1 μM-10 nM concentration range and a limit-of-detection (LOD) of 208 fM (FIG. 2D). The LOD was calculated using the linear regression equation of the most linear region and the limit-of-blank (LOB), which is defined as the highest signal obtained in response to a solution that is void of target analyte. This method of LOD calculation was done to take into consideration the peak current that is produced via nonspecific interactions within a blank solution and was used for all subsequent protein quantification. Sensitive detection is important for biosensors owing to the low abundance of biomarkers in clinical samples. The sub-pM LOD demonstrated herein positions the proximity induced bio-barcode assay as a platform for ultrasensitive protein detection.

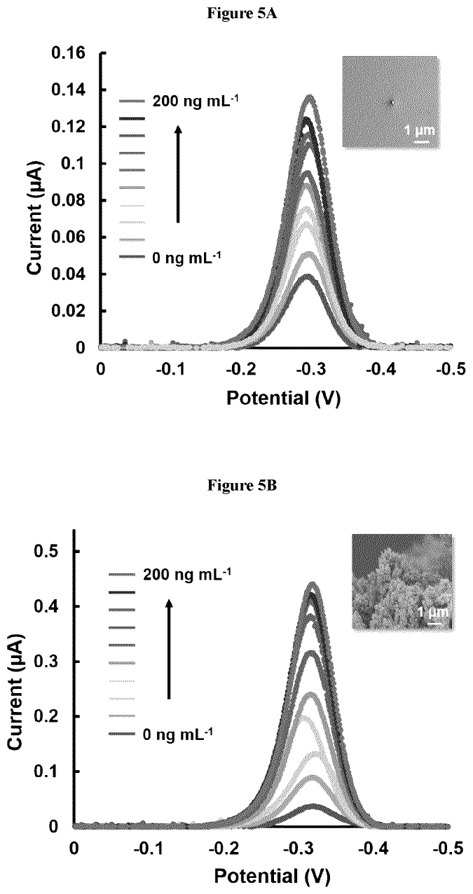

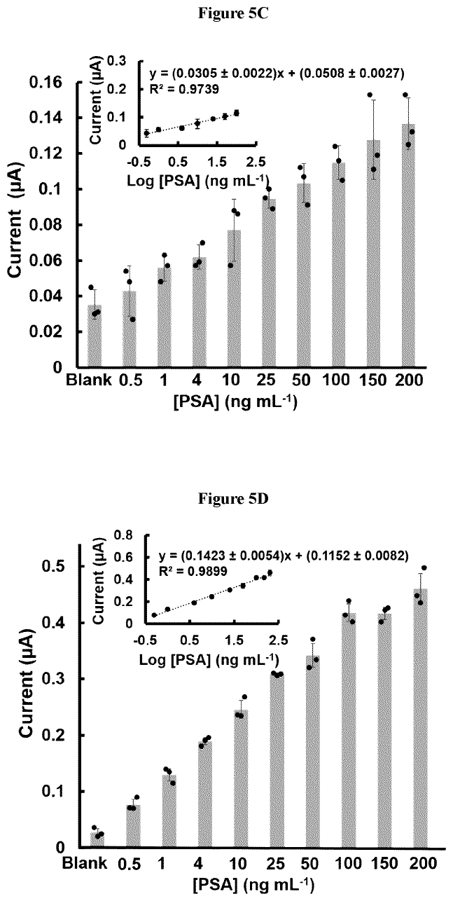

In order to demonstrate the applicability of the sensor to detecting relevant cancer protein biomarkers, prostate specific antigen (PSA) was employed as the target protein and polyclonal anti-PSA antibodies were conjugated to TB and B*C motifs, using the biotin/streptavidin interaction (FIG. 4), as biorecognition elements. To prepare the biorecognition motifs, TB and B*C, commercially-available biotinylated oligonucleotides were first conjugated to streptavidin followed by conjugation to a commercially available biotinylated antibody. The probe was then saturated with a biotin solution to block all other binding sites on the streptavidin molecule. Upon binding, the DNA-antibody/protein complexes are generated, and the programmed strand displacement reactions are initiated, leading to the release and subsequent on-chip capture of the MB-labelled reporter barcode. As a result, the measured electrochemical current on planar electrodes is increased with increasing PSA concentration (FIG. 5A), indicating the successful detection of PSA. Using data gathered from over 30 chips, PSA was analyzed within a log-linear range of 0.5 to 100 ng mL⁻¹ (y=(0.0305±0.0022)x+(0.0508±0.0027), R²=0.97) with an LOD of 2 ng mL⁻¹ (FIG. 5C). This lower LOD compared to the example with streptavidin is expected to be related to the lower binding affinity of anti-PSA and PSA compared to streptavidin and biotin, and the increased steric hindrance caused by larger biorecognition elements used for PSA. In clinical applications, PSA concentrations higher than a threshold level of 4 ng mL⁻¹ are indicative of prostate cancer. Although the achieved LOD was below this clinically-relevant threshold, the transducers were re-engineered for further enhancing their LOD for more robust and reliable sensing performance.

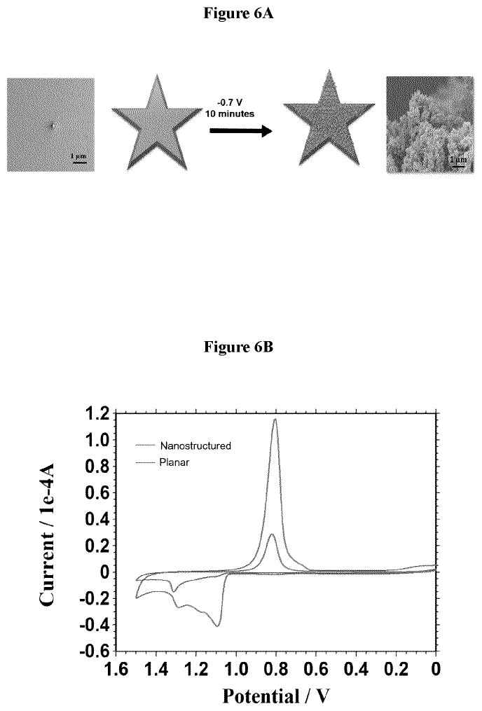

Three-dimensional transducers created from the assembly of nanostructured building blocks allow for an increased number of biorecognition probes to be deposited on the electrode surface with a more suitable orientation and spacing for target capture compared to two-dimensional sensing electrodes. Additionally, it is expected that the bulky biomolecular complexes used in this assay accumulate at the electrode surface, making it important to develop strategies for reducing steric hindrance at the surface. As a result, it was tested if performing the e-biobarcode assay on three-dimensional and nanostructured transducers would enhance the efficiency of interfacial DNA strand displacement reactions and assay sensitivity. Therefore, star-shaped gold electrodes with sharp edges were designed to result in three-dimensional nanostructured electrodes (3D nano-electrodes) following electrodeposition (FIG. 6). The electroactive surface area of a gold electrode can be found using the integration of the gold oxide reduction peak using the following formula: Γ=A/(v·482 μC cm⁻²) where Γ is the electrochemical surface area, A is the cathodic peak area in the cyclic voltammogram and v is the scan rate. Substituting in values obtained from the plot by reversibly scanning from 0-1.6 V (against Ag/AgCl) in 0.5 M H₂SO₄ at a scan rate of 0.1 V/s, the surface area of the planar electrodes was found to be 0.26 cm² and the surface of the 3D-nano electrodes was found to be 1.13 cm², roughly demonstrating a 4.5× enhancement of surface area.

The performance of the 3D nano-electrodes with planar electrodes (FIG. 5C and FIG. 5D) in analysing PSA using the e-biobarcode assay. The 3D nano-electrodes demonstrated PSA detection within the clinically-relevant log-linear range of 0.5 ng mL⁻¹-200 ng mL⁻¹ (y=(0.1423±0.0054)x+(0.1152±0.0082), R²=0.99) with a sensitivity of 0.14 μA/log (ng mL⁻¹) and a LOD of 0.3 ng mL⁻¹. Using the 3D nano-electrodes resulted in a remarkable enhancement in sensitivity (four times) and LOD (six times) compared to the planar electrodes, leading to an assay that is suitable for clinical analysis.

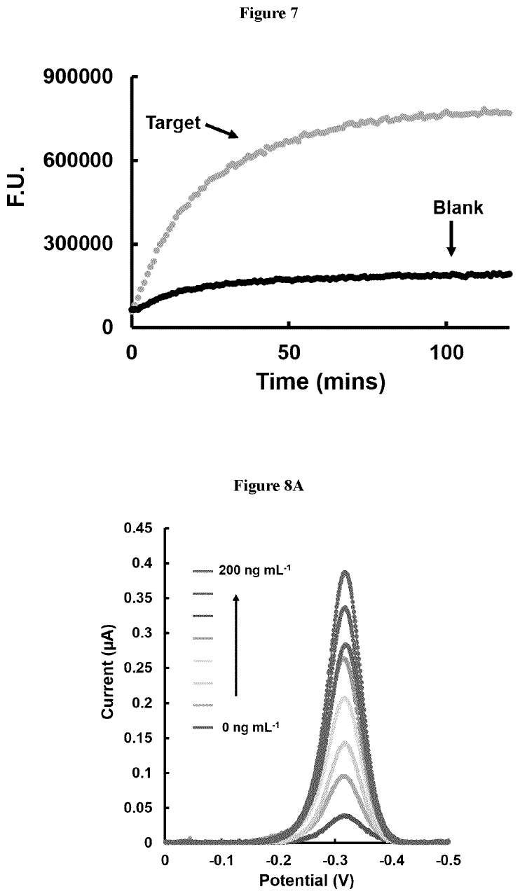

For further verification that PSA could be detected using the bio-barcode assay, a fluorescence assay was done following the same procedure that was performed for the validation assay using a streptavidin target (FIG. 7). The biotinylated TB and B*C motifs were conjugated with anti-PSA to be used as the biorecognition element and 1 μg mL⁻¹ PSA was used as the protein target. The fluorescent signal was measured in the presence and absence of PSA and an increase in signal can be seen correlating to the presence of the protein target.

For a biosensor to be used in clinical analysis and decision making, it must perform successfully in complex solutions such as serum, plasma, blood, or urine. These solutions are composed of proteins and other large biomolecules that can degrade assay reagents and/or non-specifically adsorb onto the electrode surface and influence the sensor's performance. To circumvent these effects, surface blockers such as bovine serum albumin (BSA), short chain alkanethiols, poly(ethylene glycol), carbo-free blocking solution, and gelatin have been used. In this assay, poly-A strands were used to exploit the strong affinity between the adenine bases of DNA and gold to reduce the surface area of the unreacted electrode available for non-specific adsorption of interfering biomolecules. Unlike bulky proteins used as surface blockers, the small size of poly-A strands does not interfere with electron transport or the hybridization of the capture and reporter probes while reducing the negative effects of non-specific adsorption.

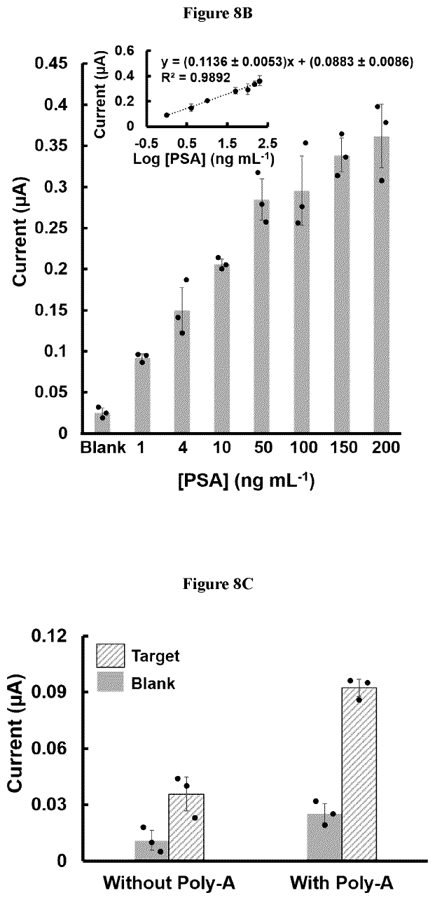

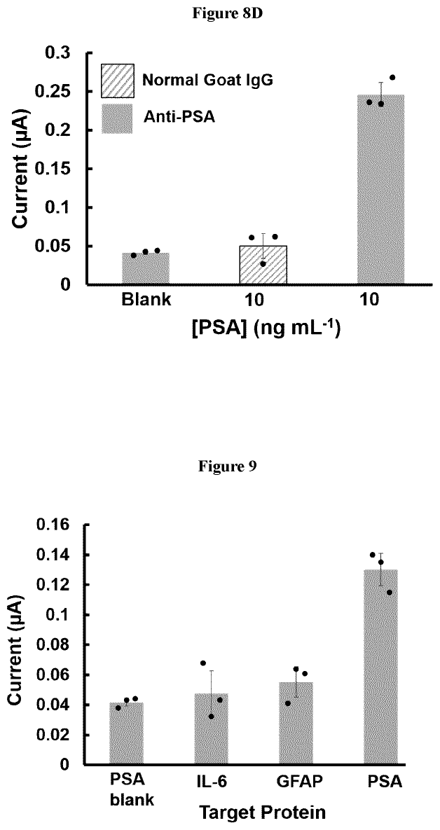

To assess whether this bio-barcode assay integrated with poly-A as a surface blocker was suitable for clinical use, the system was challenged with samples that contained PSA spiked in undiluted human plasma. As previously observed with PSA targets suspended in buffer, increasing the target concentration resulted in an increase in the electrochemical current (FIG. 8A and FIG. 8B). Although the signal magnitudes decreased from the values measured in buffer, the sensitivity remained high, achieving an LOD of 0.4 ng mL⁻¹ and a linear range in the log concentration from 1 ng mL⁻¹-200 ng mL⁻¹ (y=(0.1136±0.0053)x+(0.0883±0.0085), R²=0.99) (FIG. 8B). The loss in signal magnitude can be explained by the non-specific adsorption of large proteins and other molecules present in undiluted plasma. Nevertheless, the poly-A played a role in maintaining the signal-to-blank ratio and as a result, the assay sensitivity and LOD (FIG. 8C).

To further demonstrate the specificity of the bio-barcode assay, normal goat IgG control antibodies were used in place of anti-PSA as the detection element. As expected, a much larger current increase was seen with anti-PSA compared to the non-specific normal goat IgG antibodies (FIG. 8D). Furthermore, the sensor was challenged with non-specific proteins IL-6 and GFAP, with anti-PSA antibodies as recognition molecules (FIG. 9). Detection of these proteins was performed following the same protocol that was used for detecting PSA. Again, a statistically significant signal increase was only observed when PSA was present in solution with the specific antibody (p=0.316 for IL-6, p=0.097 for GFAP, p=0.003 for PSA). These results confirm that the assay is both specific and sensitive in real human samples and demonstrate the potential use in clinical applications.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### REFERENCES

[1] F. Li, Y. Lin, X. C. Le, Anal. Chem 2013, 85, 10835-10841.

1. A biosensor for detecting a target analyte in a sample comprising:
a) a double-stranded oligonucleotide comprising an overhang on a first strand of the oligonucleotide, and a second strand of the oligonucleotide that is a reporter moiety comprising a detectable label;
b) a first detection probe comprising a recognition moiety and a junction forming moiety, wherein the junction forming moiety comprises a first portion capable of binding by complementarity to the overhang of the first strand of the double-stranded oligonucleotide;
c) a second detection probe comprising a recognition moiety and a junction forming moiety, wherein the junction forming moiety comprises a first portion capable of binding by complementarity to an internal segment of the first strand of the double-stranded oligonucleotide; and
d) a capture probe functionalized on an electrode, wherein the capture probe comprises an immobilized strand attached to the electrode, and optionally a displaceable strand binding to the immobilized strand by partial complementarity;
wherein the junction forming moiety of the first detection probe comprises a second portion complementary to a second portion of the junction forming moiety of the second detection probe,
wherein in the presence of the target analyte, i) the recognition moiety of the first detection probe is capable of binding to the target analyte, and ii) the recognition moiety of the second detection probe binds is capable of binding to a different portion of the target analyte, whereby the binding in i) and ii) is capable of bringing the first detection probe and the second detection probe into proximity sufficient to allow for the second portion of the first detection probe to bind by complementarity to the second portion of the second detection probe, whereby the first detection probe and the second detection probe are capable of forming a stable duplex,
wherein in the presence of the target analyte, the first portion of the first detection probe is capable of binding by complementarity to the overhang of the first strand of the double-stranded oligonucleotide, and the first portion of the second detection probe is capable of binding by complementarity to the internal segment of the first strand of the double-stranded oligonucleotide, whereby the binding of the first detection probe and the second detection probe to the first strand of the double-stranded oligonucleotide is capable of releasing the reporter moiety from the double-stranded oligonucleotide in a), and
wherein the released reporter moiety is capable of binding by complementarity to the immobilized strand of the capture probe attached to the electrode, whereby the binding is capable of displacing the optional displaceable strand and bringing the detectable label on the reporter moiety close to the electrode surface for producing a detectable electrochemical signal.

2. The biosensor of claim 1, wherein the recognition moiety of the first detection probe and the recognition moiety of the second detection probe, each independently, comprises a nucleic acid, a small molecule, a peptide, or a protein, optionally wherein the protein is an antibody or an antigen-binding fragment thereof.

3. (canceled)

4. The biosensor of claim 1, wherein the junction forming moiety of the first detection probe and the junction forming moiety of the second detection probe, each independently, comprises a nucleic acid.

5. The biosensor of claim 1, wherein the immobilized strand of the capture probe and the displaceable strand of the capture probe, each independently, comprises a nucleic acid.

6. The biosensor of claim 1, wherein the detectable label comprises a redox species or photoelectrochemical species, optionally wherein the redox species is selected from the group consisting of methylene blue, methylene blue succinimide, methylene blue maleimide, Atto MB2 maleimide, other methylene blue derivatives, 3,7-Bis-[(2-Ammoniumethyl) (methyl)amino]phenothiazin-5-ium trifluoroacetate, 3,7-Bis-(piperazin-4-ium-1-yl)phenothiazin-5-ium trifluoroacetate, 3,7-Bis-[(2-ammoniumethyl)(methyl)amino] phenothiazin-5-ium chloride, 3,7-Bis-(piperazin-4-ium-1-yl)phenothiazin-5-ium chloride, and ferrocene.

7-9. (canceled)

10. The biosensor of claim 1, wherein the detectable electrochemical signal is a change in current, voltage or impedance, optionally wherein the detectable electrochemical signal is an increase in current compared to in the absence of the target analyte.

11. (canceled)

12. The biosensor of claim 1, wherein the electrode comprises a conductive material, a semi-conductive material, a metal, a metal alloy, a metal oxide, a superconductor, a semi-conductor, a carbon-based material, a conductive polymer, or combinations thereof.

13-14. (canceled)

15. The biosensor of claim 1, wherein the electrode comprises three-dimensional nanostructures.

16. The biosensor of claim 1, further comprising a surface blocker functionalized on the electrode, optionally wherein the surface blocker comprises a Poly-A oligonucleotide and/or mercaptohexanol.

17-18. (canceled)

19. The biosensor of claim 1, further comprising a counter electrode and/or a reference electrode.

20. The biosensor of claim 1, wherein the sample is an aqueous solution.

21. The biosensor of claim 1, wherein the target analyte is a protein, optionally prostate specific antigen.

22. (canceled)

23. The biosensor of claim 1, wherein the biosensor is for use in clinical and agricultural diagnostics, agri-food quality control, environmental monitoring, health screening, health monitoring, and/or pharmaceutical development.

24. A method of detecting a target analyte in a sample using the biosensor of claim 1, the method comprising:
a) mixing, optionally in a solution, the sample with the double-stranded oligonucleotide, the first detection probe and the second detection probe from the biosensor, to provide a mixture, wherein upon the mixing in the presence of the target analyte, the recognition moiety of the first detection probe binds to the target analyte, the recognition moiety of the second detection probe binds to a different portion of the target analyte, thereby bringing the first detection probe and the second detection probe into close proximity sufficient to allow for the second portion of the first detection probe to bind by complementarity to the second portion of the second detection probe, thereby forming a stable duplex, and wherein upon the forming of the stable duplex, the first portion of the first detection probe binds by complementarity to the overhang of the first strand of the double-stranded oligonucleotide, and the first portion of the second detection probe binds by complementarity to the internal segment of the first strand of the double-stranded oligonucleotide, thereby forming a junction and releasing the reporter moiety from the double-stranded oligonucleotide;
b) contacting the mixture with the capture probe functionalized on the electrode, wherein upon the contacting, in the presence of the target analyte, the released reporter moiety binds to the immobilized strand of the capture probe, optionally if the displaceable strand is present, displacing the displaceable strand, and bringing the detectable label on the reporter moiety close to the electrode surface, thereby producing a detectable electrochemical signal; and
c) measuring the detectable electrochemical signal produced from the electrode.

25. The method of claim 24, wherein the detectable electrochemical signal is a change in current, voltage or impedance in the presence of the target analyte compared to in the absence of the target analyte, optionally wherein the detectable electrochemical signal is an increase in current compared to in the absence of the target analyte, optionally wherein the target analyte is a protein, optionally prostate specific antigen.

26-29. (canceled)

30. A kit for detecting a target analyte in a sample, wherein the kit comprises:
a) a double-stranded oligonucleotide comprising an overhang on a first strand of the oligonucleotide, and a second strand of the oligonucleotide that is a reporter moiety comprising a detectable label;
b) a first detection probe comprising a recognition moiety and a junction forming moiety, wherein the junction forming moiety comprises a first portion capable of binding by complementarity to the overhang of the first strand of the double-stranded oligonucleotide;
c) a second detection probe comprising a recognition moiety and a junction forming moiety, wherein the junction forming moiety comprises a first portion capable of binding by complementarity to an internal segment of the first strand of the double-stranded oligonucleotide; and
d) instructions for use.

31. The kit of claim 30, further comprising a capture probe functionalized on an electrode, wherein the capture probe comprises an immobilized strand attached to the electrode, and optionally a displaceable strand bound to the immobilized strand by partial complementarity, and optionally further comprising at least one of a solution, a sample collector, a liquid dropper, a lancet, a bandage, gloves, and a mask.

32. (canceled)

33. A biosensor for detecting a target analyte in a sample comprising:
a) a double-stranded oligonucleotide comprising an overhang on a first strand of the oligonucleotide, and a second strand of the oligonucleotide that is a reporter moiety;
b) a first detection probe comprising a recognition moiety and a junction forming moiety, wherein the junction forming moiety comprises a first portion capable of binding by complementarity to the overhang of the first strand of the double-stranded oligonucleotide;
c) a second detection probe comprising a recognition moiety and a junction forming moiety, wherein the junction forming moiety comprises a first portion capable of binding by complementarity to an internal segment of the first strand of the double-stranded oligonucleotide; and
d) a capture probe, optionally functionalized on a solid support, wherein the capture probe comprises a signaling strand and a displaceable strand bound to the signaling strand by partial complementarity, wherein the signaling strand comprises a quenchable detectable label and the displaceable strand comprises a quencher in sufficiently close proximity to and capable of quenching the quenchable detectable label;
wherein the junction forming moiety of the first detection probe comprises a second portion complementary to a second portion of the junction forming moiety of the second detection probe,
wherein in the presence of the target analyte, i) the recognition moiety of the first detection probe is capable of binding to the target analyte, and ii) the recognition moiety of the second detection probe is capable of binding to a different portion of the target analyte, whereby the binding in i) and ii) is capable of bringing the first detection probe and the second detection probe into proximity sufficient to allow for the second portion of the first detection probe to bind by complementarity to the second portion of the second detection probe, whereby the first detection probe and the second detection probe are capable of forming a stable duplex,
wherein in the presence of the target analyte, the first portion of the first detection probe is capable of binding by complementarity to the overhang of the first strand of the double-stranded oligonucleotide, and the first portion of the second detection probe is capable of binding by complementarity to the internal segment of the first strand of the double-stranded oligonucleotide, whereby the binding of the first detection probe and the second detection probe to the first strand of the double-stranded oligonucleotide is capable of releasing the reporter moiety from the double-stranded oligonucleotide in a), and
wherein the released reporter moiety is capable of binding by complementarity to the signaling strand of the capture probe, whereby the binding is capable of displacing the displaceable strand, and
wherein the displacing is capable of distancing the quencher on the displaceable strand from the quenchable detectable label on the signaling strand, and allowing the detectable label to produce a detectable signal.

34. The biosensor of claim 33, wherein the quenchable detectable label is a fluorophore, optionally fluorescein, rhodamine, Oregon green, eosin, Texas red, cyanine, indocarbocyanine, oxacarbocyanine, thiacarbocyanine, merocyanine, dansyl, pyridyloxazole, nitrobenzoxadiazole, benzoxadiazole, anthraquinone, cascade blue, Nile red, Nile blue, cresyl violet, oxazine 170, proflavin, acridine orange, acridine yellow, auramine, crystal violet, malachite green, porphin, phthalocyanine, bilirubin, BODIPY, aza-BODIPY 29, or a derivative thereof.

35. The biosensor of claim 33, wherein the quencher is [4-((4-(dimethylamino)phenyl)azo)benzoic acid] (DABCYL acid), a fluorescence resonance energy transfer (FRET), optionally a Black Hole Quencher (BHQ) or a QSY quencher, a dinitrobenzene quencher, a Qxl quencher, Iowa Black FQ, Iowa Black RQ, IRDye QC-1, or a derivative thereof.

36. (canceled)


SEQUENCE LISTING (9 sequences)

SEQ ID NO: 1 - DNA, 32 bp, Artificial Sequence
  Synthetic construct
tttttttttttttttgtgaggttcgtgtgatg

SEQ ID NO: 2 - DNA, 41 bp, Artificial Sequence
  Synthetic construct
aagcgtgtatcccatgtgtccctcacttttttttttttttt

SEQ ID NO: 3 - DNA, 29 bp, Artificial Sequence
  Synthetic construct
catcacacggacacatgggatacacgctt

SEQ ID NO: 4 - DNA, 38 bp, Artificial Sequence
  Synthetic construct
tcttccaatcagtctctcaagcgtgtatcccatgtgtc

SEQ ID NO: 5 - DNA, 20 bp, Artificial Sequence
  Synthetic construct
tcttccaatcagtctctcaa

SEQ ID NO: 6 - DNA, 27 bp, Artificial Sequence
  Synthetic construct
tacacgcttgagagactgattggaaga

SEQ ID NO: 7 - DNA, 22 bp, Artificial Sequence
  Synthetic construct
aaaaaaaaaaaaaaaaaaaaaa

SEQ ID NO: 8 - DNA, 27 bp, Artificial Sequence
  Synthetic construct
tacacgcttgagagactgattggaaga

SEQ ID NO: 9 - DNA, 20 bp, Artificial Sequence
  Synthetic construct
tcttccaatcagtctctcaa